SEQUENCE LISTING (49 sequences)

SEQ ID NO: 1 - protein, 8 aa, Artificial Sequence
  Synthetic Peptide
DVEXNPGP

SEQ ID NO: 2 - protein, 8 aa, Artificial Sequence
  Synthetic Peptide
DIEXNPGP

SEQ ID NO: 3 - protein, 18 aa, Artificial Sequence
  Synthetic Peptide
EGRGSLLTCGDVEENPGP

SEQ ID NO: 4 - protein, 19 aa, Artificial Sequence
  Synthetic Peptide
QGWVPDLTVDGDVESNPGP

SEQ ID NO: 5 - protein, 20 aa, Artificial Sequence
  Synthetic Peptide
GGGQKDLTQDGDIEPSNPGP

SEQ ID NO: 6 - protein, 4 aa, Artificial Sequence
  Synthetic Peptide
WGXG

SEQ ID NO: 7 - protein, 4 aa, Artificial Sequence
  Synthetic Peptide
RXXR

SEQ ID NO: 8 - protein, 4 aa, Artificial Sequence
  Synthetic Peptide
RXKR

SEQ ID NO: 9 - protein, 4 aa, Artificial Sequence
  Synthetic Peptide
RXRR

SEQ ID NO: 10 - protein, 5 aa, Artificial Sequence
  Synthetic Peptide
DDDDK

SEQ ID NO: 11 - protein, 4 aa, Artificial Sequence
  Synthetic Peptide
IEGR

SEQ ID NO: 12 - protein, 4 aa, Artificial Sequence
  Synthetic Peptide
IKGR

SEQ ID NO: 13 - protein, 10 aa, Artificial Sequence
  Synthetic Peptide
EQKLISEEDL

SEQ ID NO: 14 - protein, 6 aa, Artificial Sequence
  Synthetic Peptide
HHHHHH

SEQ ID NO: 15 - protein, 9 aa, Artificial Sequence
  Synthetic Peptide
YPYDVPDYA

SEQ ID NO: 16 - protein, 11 aa, Artificial Sequence
  Synthetic Peptide
YTDIEMNRLGK

SEQ ID NO: 17 - protein, 11 aa, Artificial Sequence
  Synthetic Peptide
QPELAPEDPED

SEQ ID NO: 18 - protein, 14 aa, Artificial Sequence
  Synthetic Peptide
GKPIPNPLLGLDST

SEQ ID NO: 19 - protein, 8 aa, Artificial Sequence
  Synthetic Peptide
DYKDDDDK

SEQ ID NO: 20 - DNA, 13 bp, Artificial Sequence
  Synthetic Nucleotide
ggccnnnnnggcc

SEQ ID NO: 21 - DNA, 33 bp, Artificial Sequence
  Synthetic Nucleotide
gtcgtcaagcttgacatggacatgagggccccc

SEQ ID NO: 22 - DNA, 60 bp, Artificial Sequence
  Synthetic Nucleotide
ctccacgtcaccgcatgttagaagacttcctctgccctcacagtcacccctattgaagct

SEQ ID NO: 23 - DNA, 59 bp, Artificial Sequence
  Synthetic Nucleotide
tcttctaacatgcggtgacgtggaggagaatcccggccctatggagactgggctgcgct

SEQ ID NO: 24 - DNA, 39 bp, Artificial Sequence
  Synthetic Nucleotide
ataagaatgcggccgctatcatttacccggagagcggga

SEQ ID NO: 25 - DNA, 2133 bp, Artificial Sequence
  Synthetic Nucleotide
atggacatgagggcccccactcagctgctggggctcctgctgctctggctcccaggtgccacatttgcccaagtgctgacccagactccatcccccgtgtctgcagctgtgggaaacacagtcaccatcaattgccaggccagtcagagtgttcgtgataataactacttatcctggtatcagcagaaaccaggtcagcctcccaagctcctgatctacagggcatccactctggaatctggggtcccatcgcggtttaaaggcaatggatctgggacacaattcactctcaccatcagcgacctggagtgtgacgatgctgccacttactattgtcaaggcggttatggtgggaatttttttcctttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgtgagggcagaggaagtcttctaacatgcggtgacgtggaggagaatcccggccctatggagactgggctgcgctggcttctcctggtcgctgtgctcaaaggtgtccagtgtcagtcggtggaggagtccgggggtcgcctggtcaagcctgacgaaaccctgacaatcacctgcacagtctctggaatcgacctcaataacaatgcaatgggctgggtccgccaggctccaggggaggggctggaatacatcggattcattggtgggagtggtgccacatactactcgacctgggcgaaaggccggttcaccatctccaagtcctcgaccacggtggatctgatgatcaccagtccgacaaccgaggacacggccacctatttctgtgccagatatgctggtagtggttcttttgacttctcgggcccaggcaccctggtcaccgtctccttggggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcattttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 26 - protein, 710 aa, Artificial Sequence
  Synthetic Peptide
MDMRAPTQLLGLLLLWLPGATFAQVLTQTPSPVSAAVGNTVTINCQASQSVRDNNYLSWYQQKPGQPPKLLIYRASTLESGVPSRFKGNGSGTQFTLTISDLECDDAATYYCQGGYGGNFFPFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDCEGRGSLLTCGDVEENPGPMETGLRWLLLVAVLKGVQCQSVEESGGRLVKPDETLTITCTVSGIDLNNNAMGWVRQAPGEGLEYIGFIGGSGATYYSTWAKGRFTISKSSTTVDLMITSPTTEDTATYFCARYAGSGSFDFSGPGTLVTVSLGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 27 - protein, 714 aa, Artificial Sequence
  Synthetic Peptide
MDMRAPTQLLGLLLLWLPGATFAQVLTQTPSPVSAAVGNTVTINCQASQSVRDNNYLSWYQQKPGQPPKLLIYRASTLESGVPSRFKGNGSGTQFTLTISDLECDDAATYYCQGGYGGNFFPFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDCRXRREGRGSLLTCGDVEENPGPMETGLRWLLLVAVLKGVQCQSVEESGGRLVKPDETLTITCTVSGIDLNNNAMGWVRQAPGEGLEYIGFIGGSGATYYSTWAKGRFTISKSSTTVDLMITSPTTEDTATYFCARYAGSGSFDFSGPGTLVTVSLGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 28 - DNA, 711 bp, Artificial Sequence
  Synthetic Nucleotide
atggacatgagggcccccactcagctgctggggctcctgctgctctggctcccaggtgccacatttgcccaagtgctgacccagactccatcccccgtgtctgcagctgtgggaaacacagtcaccatcaattgccaggccagtcagagtgttcgtgataataactacttatcctggtatcagcagaaaccaggtcagcctcccaagctcctgatctacagggcatccactctggaatctggggtcccatcgcggtttaaaggcaatggatctgggacacaattcactctcaccatcagcgacctggagtgtgacgatgctgccacttactattgtcaaggcggttatggtgggaatttttttcctttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 29 - DNA, 1371 bp, Artificial Sequence
  Synthetic Nucleotide
atggagactgggctgcgctggcttctcctggtcgctgtgctcaaaggtgtccagtgtcagtcggtggaggagtccgggggtcgcctggtcaagcctgacgaaaccctgacaatcacctgcacagtctctggaatcgacctcaataacaatgcaatgggctgggtccgccaggctccaggggaggggctggaatacatcggattcattggtgggagtggtgccacatactactcgacctgggcgaaaggccggttcaccatctccaagtcctcgaccacggtggatctgatgatcaccagtccgacaaccgaggacacggccacctatttctgtgccagatatgctggtagtggttcttttgacttctcgggcccaggcaccctggtcaccgtctccttggggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcattttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 30 - DNA, 24 bp, Artificial Sequence
  Synthetic Nucleotide
gacgtggaggagaatcccggccct

SEQ ID NO: 31 - DNA, 44 bp, Artificial Sequence
  Synthetic Nucleotide
agtggatccgacgtggaggagaatcccggccctatggagactgg

SEQ ID NO: 32 - DNA, 25 bp, Artificial Sequence
  Synthetic Nucleotide
taggctgcagtcatttacccggaga

SEQ ID NO: 33 - DNA, 22 bp, Artificial Sequence
  Synthetic Nucleotide
gggaagcttatggacatgaggg

SEQ ID NO: 34 - DNA, 22 bp, Artificial Sequence
  Synthetic Nucleotide
ggctgcagacagtcacccctat

SEQ ID NO: 35 - protein, 8 aa, Artificial Sequence
  Synthetic Peptide
DVEENPGP

SEQ ID NO: 36 - DNA, 24 bp, Artificial Sequence
  Synthetic Nucleotide
gacgtcgaagagaacccagggccc

SEQ ID NO: 37 - DNA, 12 bp, Artificial Sequence
  Synthetic Nucleotide
gaagagaaccca

SEQ ID NO: 38 - DNA, 33 bp, Artificial Sequence
  Synthetic Nucleotide
gtcgtcaagcttatgagggcccctgctcagatt

SEQ ID NO: 39 - DNA, 60 bp, Artificial Sequence
  Synthetic Nucleotide
ctccacgtcaccgcatgttagaagacttcctctgccctcacactcattcctgttgaagct

SEQ ID NO: 40 - DNA, 61 bp, Artificial Sequence
  Synthetic Nucleotide
tcttctaacatgcggtgacgtggaggagaatcccggccctatggcttgggtgtggaccttg

SEQ ID NO: 41 - DNA, 39 bp, Artificial Sequence
  Synthetic Nucleotide
ataagaatgcggccgctatcatttaccaggagagtggga

SEQ ID NO: 42 - DNA, 2127 bp, Artificial Sequence
  Synthetic Nucleotide
atgagggcccctgctcagattcttggcttcttgttgctctggtttccaggtattagatgtgacatcaagatgacccagtcgccatcctccttatctgcctctctgggagaaagagtcagtctcacttgtcgggcaagtcaggaaattagtggttacttaagctggcttcagcaaaaaccagatggaactgttaaacgcctgatctacgccgcatccactttacattctggtgtcccaaaaaggttcagtggcagtaggtctgggtcagactattctctcaccatcagcagccttgagtctgacgattttgcagactattactgtctacagtatgctagtgatccgtggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccatcgtcaagagcttcaacaggaatgagtgtgagggcagaggaagtcttctaacatgcggtgacgtggaggagaatcccggccctatggcttgggtgtggaccttgctattcctgatggcagctgcccaaagtatccaagcacagatccagttggtgcagtctggacctgagctgaagaagcctggagagacagtcaagatctcctgcaagacttctggttataccttcacagactattcaatgcactgggtgaagcaggctccaggaaagggtttaaagtggatgggctggataaacactgagactggtgggccaacatatgcagatgacttcaagggacggtttgccttctctttggaaacctctgccagcactgcctatttgcagatcaacaacctcaaaaatgaggacacggctacatatttctgtgctagaacttccgtctactggggccaaggcaccactctcacagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagccagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgaaaccccgggaggagcagatcaacagcactttccgttcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacaaacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 43 - protein, 708 aa, Artificial Sequence
  Synthetic Peptide
MRAPAQILGFLLLWFPGIRCDIKMTQSPSSLSASLGERVSLTCRASQEISGYLSWLQQKPDGTVKRLIYAASTLHSGVPKRFSGSRSGSDYSLTISSLESDDFADYYCLQYASDPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECEGRGSLLTCGDVEENPGPMAWVWTLLFLMAAAQSIQAQIQLVQSGPELKKPGETVKISCKTSGYTFTDYSMHWVKQAPGKGLKWMGWINTETGGPTYADDFKGRFAFSLETSASTAYLQINNLKNEDTATYFCARTSVYWGQGTTLTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 44 - DNA, 32 bp, Artificial Sequence
  Synthetic Nucleotide
gtcgtcaagcttatggaaaccccagcgccagt

SEQ ID NO: 45 - DNA, 60 bp, Artificial Sequence
  Synthetic Nucleotide
ctccacgtcaccgcatgttagaagacttcctctgccctcgcactctcccctgttgctctt

SEQ ID NO: 46 - DNA, 99 bp, Artificial Sequence
  Synthetic Nucleotide
ataagaatgcggccgctactatttacccggagacagggatcttctaacatgcggtgacgtggaggagaatcccggccctatggactgcacctggaggat

SEQ ID NO: 47 - DNA, 39 bp, Artificial Sequence
  Synthetic Nucleotide
ataagaatgcggccgctactatttacccggagacaggga

SEQ ID NO: 48 - DNA, 2202 bp, Artificial Sequence
  Synthetic Nucleotide
atggaaaccccagcgccagttctcttcctcctgctactctggctcccagataccggagacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccccctacacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgcgagggcagaggaagtcttctaacatgcggtgacgtggaggagaatcccggccctatggactgcacctggaggatcctcctcttggtggcagcagctacaggcacccacgccgaggtccaggtgcagctggtggagtctgggggaggcttggtccagcctggggggtccctgagactctcctgtgcagcctctggattcacctttagtgattattggatgagctgggtccgccaggctccagggaaggggctggagtgggtggcccacataaagcaagatggaagtgagaaatactatgtggactctgtgaagggccgattcaccatctcccgagacaaagccaagaactcactgtatctccaaatgaacagcctgagagccgaggacacggccgtgtattactgtgcgaggtgtccggtgcgggaacgtgactggtaccgtgcgcgtggggagtactactacgtctacatggacgtctggggcaaggggaccacggtcaccgtctcctcagcttccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatag

SEQ ID NO: 49 - protein, 733 aa, Artificial Sequence
  Synthetic Peptide
METPAPVLFLLLLWLPDTGDIQMTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEGRGSLLTCGDVEENPGPMDCTWRILLLVAAATGTHAEVQVQLVESGGGLVQPGGSLRLSCAASGFTFSDYWMSWVRQAPGKGLEWVAHIKQDGSEKYYVDSVKGRFTISRDKAKNSLYLQMNSLRAEDTAVYYCARCPVRERDWYRARGEYYYVYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2011/0045534 A1
(43) Pub. Date: Feb. 24, 2011

(54) Nucleic Acid Cassette For Producing Recombinant Antibodies

(75) Inventors: WAN CHEUNG CHEUNG, LEXINGTON, MA (US); SHUJI SATO, SOMERVILLE, MA (US); ROBERTO POLAKIEWICZ, LEXINGTON, MA (US)

Correspondence Address: Nancy Chiu Wilker, Ph.D.;Chief Intellectual Property Counsel, CELL SIGNALING TECHNOLOGY, INC., 3 Trask Lane, Danvers, MA, 01923, US

(73) Assignee: CELL SIGNALING TECHNOLOGY, INC., DANVERS, MA (US)

(21) Appl. No.: 12/624,329

(22) Filed: Nov. 23, 2009

Related U.S. Application Data
(60) Provisional application No. 61/274,723, filed on Aug. 20, 2009.

(51) Int. Cl. C12P 21/06; C07H 21/04; C12N 15/63; C12N 5/10; C07K 16/00; C12P 19/34

(52) U.S. Cl. 435/69.6; 536/23.53; 435/320.1; 435/325; 530/387.3; 435/91.2

(57) ABSTRACT

The invention provides a nucleic acid cassette comprising components in the following structure: A-B-C, wherein “A” is a nucleic acid sequence encoding a light chain of a first antibody (or antigen binding domain thereof), “B” is a nucleic acid sequence encoding a 2A peptide, “C” is a nucleic acid sequence encoding a heavy chain of a second antibody (or antigen binding domain thereof), and “−” is a phosphodiester or phosphorothioate bond. Also provided are methods for making recombinant antibodies using the nucleic acid cassette of the invention, cells and vector comprising the nucleic acid cassette of the invention, and kits for making the nucleic acid cassette of the invention.

### RELATED APPLICATIONS

This application claims priority to U.S. Provisional Application 61/274,723, filed Aug. 20, 2009, the entire contents of which is hereby incorporated by reference.

### BACKGROUND OF THE INVENTION

The invention relates to the field of molecular biology and immunology, particularly the field of recombinant antibody production.

Antibodies have been used research tools for decades. More recently, antibodies have found use as diagnostic and therapeutic tools. For example, trastuzumab (sold by Genentech under the trademark Herceptin), an antibody that binds selectively to the HER2 protein, is FDA approved for the treatment of patients with HER2-positive breast cancer. Similarly, several antibodies have received FDA approval for use as diagnostic tools, including CEA-Scan for colorectal cancer detection, Myoscint for detecting myocardial injury, and Verluma for advanced small cell lung cancer.

However, production of antibodies by classical means (e.g., from an immortalized hybridoma cell line according to the method of Kohler and Milstein) may be hampered by the secretion rates of the antibody-producing hybridoma. Thus, efforts have been made to produce antibodies using recombinant DNA technology.

Various methods for generating recombinant antibodies are known in the art (see, e.g., U.S. Patent Publication No. 20070065912; U.S. Pat. No. 5,969,108; U.S. Pat. No. 6,331,415; U.S. Pat. No. 7,498,024; and U.S. Pat. No. 7,485,291, all of which are herein incorporated by reference in their entirety). Each of these methods (and other known in the art) has its weaknesses. Thus, there is a need for a new system to generate recombinant antibodies.

### SUMMARY OF THE INVENTION

The invention provides a genetic cassette that can be used, applying standard molecular biology and cell biology techniques, to produce a recombinant antibody.

Accordingly, in a first aspect, the invention provides a nucleic acid cassette comprising components in the following structure in a 5′ to 3′ direction on a sense strand: A-B-C, where “A” is a nucleic acid sequence encoding at least an antigen binding domain of a light chain of a first antibody, “B” is a nucleic acid sequence encoding a 2A peptide, “C” is a nucleic acid sequence encoding at least an antigen binding domain of a heavy chain of a second antibody, and “−” is a bond selected from the group consisting of a phosphodiester bond and a phosphorothioate bond. In some embodiments, the “−” is a phosphodiester bond.

In another aspect, the invention provides a nucleic acid cassette comprising components in the following structure in a 5′ to 3′ direction on a sense strand: A-B-C, wherein “A” is a nucleic acid sequence encoding a light chain of a first antibody, “B” is a nucleic acid sequence encoding a 2A peptide, “C” is a nucleic acid sequence encoding a heavy chain of a second antibody, and “−” is a bond selected from the group consisting of a phosphodiester bond and a phosphorothioate bond. In some embodiments, the “−” is a phosphodiester bond.

In some embodiments, the nucleic acid cassette further comprises components in the following structure: A!-A-B-C!-C, where “A!” is a nucleic acid sequence encoding a first leader peptide, and “C!” is a nucleic acid sequence encoding a second leader peptide. In some embodiments, the first leader peptide is a light chain leader peptide. In some embodiments, the second leader peptide is a heavy chain leader peptide.

In some embodiments, the nucleic acid cassette further comprises components in the following structure: A-B-C-D, wherein “D” is a nucleic acid sequence encoding a tag.

In yet a further embodiment, the nucleic acid cassette further comprising components in the following structure: A-p-B-C, wherein “p” is a nucleic acid sequence encoding a protease recognition site.

In a further aspect, the invention provides a nucleic acid cassette comprising components in the following structure: A-a-B-C-c, wherein “A” is a nucleic acid sequence encoding an antigen binding domain of a light chain of a first antibody, “a” is a nucleic acid sequence encoding a stem of a light chain of a second antibody, “B” is a nucleic acid sequence encoding a 2A peptide, “C” is a nucleic acid sequence encoding an antigen binding domain of a heavy chain of a third antibody, “c” is a nucleic acid sequence encoding a stem of a heavy chain of a fourth antibody, and “−” is a bond selected from the group consisting of a phosphodiester bond and a phosphorothioate bond. In some embodiments, the “−” is a phosphodiester bond.

In some embodiments, the nucleic acid cassette further comprises components in the following structure: A!-A-a-B-C!-C-c, wherein “A!” is a nucleic acid sequence encoding a first leader peptide, and “C!” is a nucleic acid sequence encoding a second leader peptide. In some embodiments, the first leader peptide is from a light chain of an antibody. In some embodiments, the second leader peptide is from a heavy chain of an antibody.

In further embodiments, the nucleic acid cassette further comprises components in the following structure: A-a-B-C-c-D, wherein “D” is a nucleic acid sequence encoding a tag.

In another embodiment, the nucleic acid cassette further comprising components in the following structure: A-a-p-B-C-c, wherein “p” is a nucleic acid sequence encoding a protease recognition site.

In various embodiments of all of the aspects of the invention, the first antibody and the second antibody are the same. In some embodiments, the third antibody and the fourth antibody are the same. In various embodiments, the “−” is a phosphodiester bond. In some embodiments, the first antibody and the second antibody are from the same species of animal. In some embodiments, the animal is a human, a mouse, a rabbit, or a rat. In some embodiments, the first antibody and the second antibody are of an isotype selected from the group consisting of IgG, IgD, IgA, IgE, and IgM.

In some embodiments, the 2A peptide comprises an amino acid sequence selected from the group consisting of DVEXNPGP (SEQ ID NO: 1) and DIEXNPGP (SEQ ID NO: 2), where X is any amino acid residue. In some embodiments, the 2A comprises an amino acid sequence of EGRGSLLTCGDVEENPGP (SEQ ID NO: 3).

In a further aspect, the invention provides a vector, such as an expression vector, comprising the nucleic acid cassette of the invention.

In another aspect, the invention provides a method for making a recombinant antibody comprising (a) introducing the nucleic acid cassette of the invention into a cell such that it is expressed by the cell; (b) maintaining the cell of step (a) in a culture media, and isolating the antibody from the cell or the culture media of step (b).

In another aspect, the invention provides a cell introduced with a nucleic acid cassette of the invention. In some embodiments, the cell expresses the nucleic acid cassette.

In a further aspect, the invention provides a recombinant antibody produced by a cell expressing a nucleic acid cassette of the invention.

In another aspect, the invention provides a kit comprising a first primer comprising a 5′ portion comprising a recognition site of a first restriction endonuclease and a 3′ portion that hybridizes to an antisense strand of a nucleic acid sequence encoding a leader peptide of a light chain of a first antibody; a second primer comprising a 5′ portion comprising a nucleic acid sequence that is complementary to a first part of a 2A-peptide encoding nucleic acid sequence and a 3′ portion that hybridizes to a nucleic acid sequence encoding a constant region of a light chain of a second antibody; a third primer comprising a 5′ portion comprising a nucleic acid sequence that encodes a second part of a 2A peptide and a 3′ portion that hybridizes to an antisense strand of a nucleic acid sequence encoding a leader peptide of a heavy chain of an third antibody; a fourth primer comprising a 5′ portion comprising a recognition site of a second restriction endonuclease (or the two sites may be recognized by the same endonuclease with interrupted palindromic recognition sites with degenerate sequences for directional cloning) and a 3′ portion that hybridizes to a nucleic acid sequence encoding a constant region of a heavy chain of a third antibody; and instructions for using the first, second, third, and fourth primers to generate a nucleic acid cassette from a sample comprising nucleic acid encoding the first antibody, the second antibody, the third antibody, and the fourth antibody.

In some embodiments, the first antibody and the second antibody are the same. In some embodiments, the third antibody and the fourth antibody are the same. In some embodiments, the first antibody, second antibody, third antibody, and fourth antibody are the same.

In some embodiments, the kit further comprises a thermostable DNA polymerase (e.g., Taq polymerase). In some embodiments, the kit further comprises a first restriction endonuclease and a second restriction endonuclease. In some embodiments, the first restriction endonuclease and the second restriction endonuclease are the same. In some embodiments, the kit further comprises a vector comprising a polylinker (also known as a multi-cloning site) comprising the first restriction endonuclease recognition site and the second restriction endonuclease recognition site. In some embodiments, the kit further comprises a vector fragment of a vector comprising a polylinker comprising the first restriction endonuclease recognition site and the second restriction endonuclease recognition site digested with the first restriction endonuclease and the second restriction endonuclease.

In a further aspect, the invention provides a method for making a nucleic acid cassette. The method comprises (a) amplifying a nucleic acid molecule encoding a light chain comprising a leader peptide and a constant region of a first antibody with a first primer comprising a 5′ portion comprising a recognition site of a first restriction endonuclease and a 3′ portion that hybridizes to an antisense strand of a nucleic acid sequence encoding the leader peptide of the light chain and a second primer comprising a 5′ portion comprising a nucleic acid sequence that is complementary to a first part of a 2A-peptide encoding nucleic acid sequence and a 3′ portion that hybridizes to a nucleic acid sequence encoding the constant region of the light chain and (b) amplifying a nucleic acid molecule encoding a heavy chain comprising a leader peptide and a constant region of a second antibody with a third primer comprising a 5′ portion comprising a nucleic acid sequence that encodes a second part of a 2A peptide and a 3′ portion that hybridizes to an antisense strand of a nucleic acid sequence encoding the leader peptide of the heavy chain and a fourth primer comprising a 5′ portion comprising a recognition site of a second restriction endonuclease and a 3′ portion that hybridizes to a nucleic acid sequence encoding the constant region of the heavy chain of a third antibody. In step (c), the products of steps (a) and (b) are mixed and amplified with the first primer and the fourth primer to generate a full-length cassette. In some embodiments, the amplifying step is by polymerase chain reaction (PCR) amplification.

### BRIEF DESCRIPTION OF THE DRAWINGS

FIG. 1 is a schematic representation depicting a non-limiting representative example of a L-2A-H nucleic acid cassette of the invention. In this example, in order from 5′ to 3′ lies nucleic acid encoding the entire light chain including the light chain leader sequence (L_{K} from the kappa light chain, although the L_{L} leader from the lambda Light chain can also be used), followed by nucleic acid encoding the 18 amino acid long T2A peptide, followed by nucleic acid encoding the entire heavy chain sequence including the heavy-chain leader sequence (L_{H}). All three segments are in frame and can be translated as a single polypeptide. Translation pauses and prematurely terminates at the glycine nearest to the C-terminal end of the 2A peptide to release the first polypeptide (i.e., the light chain with a fragment of the 2A peptide) and then restarts to complete the synthesis of the second polypeptide (i.e., the heavy chain with a P residue at its N′ terminus).

FIGS. 2A and 2B are schematic representations showing the assembly of the full-length L-2A-H cassette by a two-step PCR process. FIG. 2A shows the first step which consists of amplification of the light and heavy chains independently. The light chain is amplified with a forward primer that hybridizes to the 5′ end of the light chain leader sequence, and a reverse primer that in its 5′ end encodes the amino-terminal half of the T2A peptide in frame and hybridizes to the 3′ end of the light chain sequence. The heavy chain is amplified with a forward primer that in its 5′ end encodes the C-terminal half of the T2A peptide in-frame and hybridizes to the 5′ end of the heavy chain leader sequence, and a reverse primer that hybridizes to the 3′ end of the heavy chain sequence. FIG. 2B shows how the L-2A-H cassette is assembled in the second PCR step using Primers A and D. Primers B and C are complementary at their 5′ ends to create a nucleic acid sequence encoding the T2A peptide and therefore can hybridize to each other to act as a template for synthesis of the full-length cassette during amplification with primers A and D. The full-length cassette contains a HindIII and a NotI recognition site at its 5′ and 3′ ends, respectively, which allows the cassette to be cloned into any vector containing the same two restriction endonuclease recognition sites.

FIGS. 3A and 3B are scanned images of Western blotting analyses of intracellular (FIG. 3A) and secreted (FIG. 3B) IgG expression in HEK293T cells transfected with constructs encoding anti-HER2 and anti-MRPL11 rabbit IgG. Stars indicate the band of the full-length H-2A-L or L-2A-H translational product with a mobility of approximately 80 kDa.

FIGS. 4A and 4B are scanned images of Western blotting analyses of intracellular (FIG. 4A) and secreted (FIG. 4B) IgG expression in HEK293T cells transfected with constructs encoding anti-ERK2p, anti-MRPL11 and SUZ12 rabbit IgG. Stars indicate the band of the full-length H-2A-L or L-2A-H translational product with a mobility of approximately 80 kDa.

### DETAILED DESCRIPTION OF THE PREFERRED EMBODIMENTS

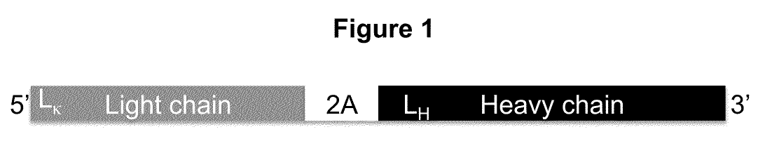

The invention provides a nucleic acid cassette that can be manipulated, using standard molecular biology and cell biology techniques, to produce a recombinant antibody. A schematic diagram of a non-limiting representative nucleic acid cassette of the invention is provided in FIG. 1.

The further aspects, advantages, and embodiments of the invention are described in more detail below. The patents, published applications, and scientific literature referred to herein establish the knowledge of those with skill in the art and are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter. As used herein, the following terms have the meanings indicated. As used in this specification, the singular forms “a,” “an” and “the” specifically also encompass the plural forms of the terms to which they refer, unless the content clearly dictates otherwise. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of recombinant DNA technology and immunology include Ausubel et al., Current Protocols in Molecular Biology, Wiley InterScience, New York, N.Y, (2007, and updates up to and including 2009), Coligan et al., Current Protocols in Immunology Wiley InterScience, New York, N.Y, (2007, and updates up to and including 2009), Lo et al., Antibody Engineering: Methods and Protocols, Humana Press, 2003; Sambrook et al., Molecular Cloning: A Laboratory Manual, 2nd Ed., Cold Spring Harbor Laboratory Press, New York (1989); Kaufman et al., Eds., Handbook of Molecular and Cellular Methods in Biology in Medicine, CRC Press, Boca Raton (1995); McPherson, Ed., Directed Mutagenesis: A Practical Approach, IRL Press, Oxford (1991). Standard reference works setting forth the general principles of pharmacology include Goodman and Gilman's The Pharmacological Basis of Therapeutics, 11th Ed., McGraw Hill Companies Inc., New York (2006).

In a first aspect, the invention provides a nucleic acid cassette comprising components in the following structure in a 5′ to 3′ direction on a sense strand: A-B-C, where “A” is a nucleic acid sequence encoding at least an antigen binding domain of a light chain of a first antibody, “B” is a nucleic acid sequence encoding a 2A peptide, “C” is a nucleic acid sequence encoding at least an antigen binding domain of a heavy chain of a second antibody, and “−” is a bond selected from the group consisting of a phosphodiester bond and a phosphorothioate bond. In some embodiments, the “−” is a phosphodiester bond.

The invention stems from the unexpected discovery that the order of the nucleic acid encoding the light chain and the nucleic acid encoding the heavy chain in the nucleic acid cassette is important to the amount and quality of the encoded antibody secreted by a cell containing the nucleic acid cassette. For example, the order of heavy and light chain could be, instead, the heavy chain-encoding nucleic acid first, then the 2A peptide-encoding nucleic acid, followed by the light chain-encoding nucleic acid. Indeed, this H-2A-L cassette is described below in the Examples and compared to the L-2A-H cassette of the invention. The presence of the 2A peptide sequence adds a series of amino acids (17 amino acids in the case of T2A peptide) to the C-terminus of the first polypeptide and only a single proline to the N-terminus of the second polypeptide. Therefore, it is logical to place the heavy chain before and the light chain after the 2A peptide sequence (i.e., in an H-2A-L order) because the heavy chain constant region 3 (at the extreme C-terminus of the heavy chain) is the furthest away from the N-terminal variable region and the hydrophobic transmembrane domain lies in the membrane-bound forms of immunoglobulins (an isoform that is expressed due to alternate splicing that changes the site of polyadenylation (see Alt et al., Cell. 20(2): 293-301, 1980; Nelson et al., Mol Cell Biol. 3(7): 1317-1332, 1983). Thus, the addition of extra amino acids from the 2A peptide would seem to be least likely to affect binding of the antibody to its cognate antigen.

Accordingly, as described below in the Examples, nucleic acid cassettes encoding four antibodies were constructed and tested in both the H-2A-L and L-2A-H format to determine whether there was a difference in expression, secretion and activity of the antibody. Surprisingly, it was found that the light chain-2A-heavy chain (L-2A-H) configuration secreted a much higher level of functional antibody than the heavy chain-2A-light chain (H-2A-L) configuration. Thus, the order of the components of light chain-encoding and heavy chain-encoding components of the nucleic acid cassette of the invention is an unexpectedly important feature of the invention.

In another aspect, the invention provides a nucleic acid cassette comprising components in the following structure in a 5′ to 3′ direction on a sense strand: A-B-C, wherein “A” is a nucleic acid sequence encoding a light chain of a first antibody, “B” is a nucleic acid sequence encoding a 2A peptide, “C” is a nucleic acid sequence encoding a heavy chain of a second antibody, and “−” is a bond selected from the group consisting of a phosphodiester bond and a phosphorothioate bond. In some embodiments, the “−” is a phosphodiester bond.

In accordance with the invention, by a “nucleic acid cassette” is meant a structure into which one or more nucleic acid sequences can be inserted or from which one or more nucleic acid sequences can be removed, where the entire cassette itself can be inserted into or removed from a vector such as a plasmid, or the genome of a cell.

The terms “nucleic acid molecule,” and “nucleic acid sequence” are used interchangeably herein to refer to polymers of nucleotides of any length, and include, without limitation, DNA, RNA, DNA/RNA hybrids, and modifications thereof. Unless otherwise specified, where the nucleotide sequence is provided, the nucleotides are set forth in a 5′ to 3′ orientation. Thus, the nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, cabamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. The nucleic acid molecules described herein may also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha-anomeric sugars, epimeric sugars such as arabinose, xyloses or lyxoses, pyranose sugars, furanose sugars, sedoheptuloses, acyclic analogs and abasic nucleoside analogs such as methyl riboside.

As used herein, by “sense” strand of a double stranded nucleic acid molecule is meant the strand that encodes for a polypeptide. Thus, the orientation of the sense strand of a DNA molecule is the same as the orientation of an mRNA molecule (where all the T residues in the DNA are replaced by U in the mRNA molecule). Similarly, by “antisense” is meant the strand of a double stranded nucleic acid molecule that is complementary to the sense strand.

It shall be understood that when a structure of a cassette is provided (e.g., A-B-C), the “−” symbol may be a phosphodiester linkage, but may also be any type of linkage to join together two nucleotides (e.g., the 3′ nucleotide of the A molecule with the 5′ nucleotide of the B molecule). One or more phosphodiester linkages may be replaced by alternative linking groups. These alternative linking groups include, but are not limited to, embodiments wherein phosphate is replaced by P(O)S (“thioate”), P(S)S (“dithioate”), “(O)NR.sub.2 (“amidate”), P(O)R, P(O)OR′, CO or CH.sub.2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a nucleic acid cassette of the invention need be identical. The preceding description applies to all nucleic acid molecules referred to herein, including RNA and DNA.

Unless otherwise indicated, each nucleotide sequence (also called a nucleic acid sequence) set forth herein is presented as a sequence of deoxyribonucleotides (abbreviated A, G, C and T). However, by “sequence” of a nucleic acid molecule is intended, for a DNA nucleic acid molecule, a sequence of deoxyribonucleotides, and for an RNA nucleic acid molecule, the corresponding sequence of ribonucleotides (A, G, C and U), where each thymidine deoxyribonucleotide (T) in the specified deoxyribonucleotide sequence is replaced by the ribonucleotide uridine (U) in the corresponding ribonucleotide sequence. For instance, reference to an RNA molecule having the sequence set forth using deoxyribonucleotide abbreviations is intended to indicate an RNA molecule having a sequence in which each deoxyribonucleotide A, G or C of a DNA sequence has been replaced by the corresponding ribonucleotide A, G or C, and each deoxyribonucleotide T has been replaced by a ribonucleotide U.

The nucleic acid cassette of the invention includes a component encoding a 2A peptide. 2A peptides, which were identified in the Aphthovirus subgroup of picornaviruses, causes a ribosomal “skip” from one codon to the next without the formation of a peptide bond between the two amino acids encoded by the codons (see Donnelly et al., J. of General Virology 82: 1013-1025 (2001); Donnelly et al., J. of Gen. Virology 78: 13-21 (1997); Doronina et al., Mol. And. Cell. Biology 28(13): 4227-4239 (2008); Atkins et al., RNA 13: 803-810 (2007)). By “codon” is meant three nucleotides on an mRNA (or on the sense strand of a DNA molecule) that are translated by a ribosome into one amino acid residue. Thus, two polypeptides can be synthesized from a single, contiguous open reading frame within an mRNA when the polypeptides are separated by a 2A oligopeptide sequence that is in frame. 2A peptides have been used to generate a recombinant, multi-chain T cell receptor (see, e.g., Szymczak et al., Nat. Biotechnol. 22: 589-594 (2004) and transgenic mice expressing a membrane-localized red fluorescent protein and nucleus-localized green fluorescent protein (see, e.g., Trichas et al., BMC Biology 6:40 (2008)).

In various embodiments, the 2A peptide comprises the amino acid sequence Asp-Val/Ile-Glu-X-Asn-Pro-Gly-Pro, where X is any amino acid residue and where translation can prematurely terminate after the glycine at the 7^{th} position and restart from the proline at the 8^{th} position. Note that in single letter code, this sequence is DVEXNPGP (SEQ ID NO: 1) or DIEXNPGP (SEQ ID NO: 2), where X is any amino acid residue and where translation can prematurely terminate after the glycine at the 7^{th} position and restart from the proline at the 8^{th} position. In some embodiments, the 2A peptide comprises the amino acid sequence QGWVPDLTVDGDVESNPGP (SEQ ID NO: 4), where translation can prematurely terminate after the glycine at the 18^{th} position and restart from the proline at the 19^{th} position. In some embodiments, the 2A peptide comprises the amino acid sequence GGGQKDLTQDGDIEPSNPGP (SEQ ID NO: 5), where translation can prematurely terminate after the glycine at the 19^{th} position and restart from the proline at the 20^{th} position. In some embodiments, the 2A peptide is from the Thosea asigna virus (and may be referred to as a T2A peptide) and comprises the amino acid sequence EGRGSLLTCGDVEENPGP (SEQ ID NO: 3), where translation can prematurely terminate after the glycine at the 17^{th} position and restart from the proline at the 18^{th} position.

The nucleic acid cassette of the invention may be flanked by sites (e.g., a restriction endonuclease recognition site) that facilitate its insertion or removal from a backbone nucleic acid molecule (e.g., a vector or the genome of a cell or animal). In some embodiments, where the cassette is flanked by restriction endonuclease sites, those sites do not occur within the cassette itself For example, some relatively rare-cutting restriction endonucleases include AscI, NotI, SfiI, NruI, MluI, SacII, BssHII, Pad, BstEII, FseI, and BstXI.

In some embodiments, where the cassette is flanked by restriction endonuclease sites, the restriction endonuclease is able to cut a linearized nucleic acid molecule close to the end. For example, BamHI, EcoRI, HindIII, MluI, NcoI, NotI, XbaI, XhoI, and PstI are some non-limiting examples of restriction endonucleases that can cut at their recognition site when the recognition site is close to the end of a linearized nucleic acid molecule. Use of such restriction endonucleases facilitates cloning of a linearized nucleic acid molecule (e.g., a PCR product) into a vector or genome of a cell or animal.

In a further aspect, the invention provides a vector, such as an expression vector, comprising the nucleic acid cassette of the invention.

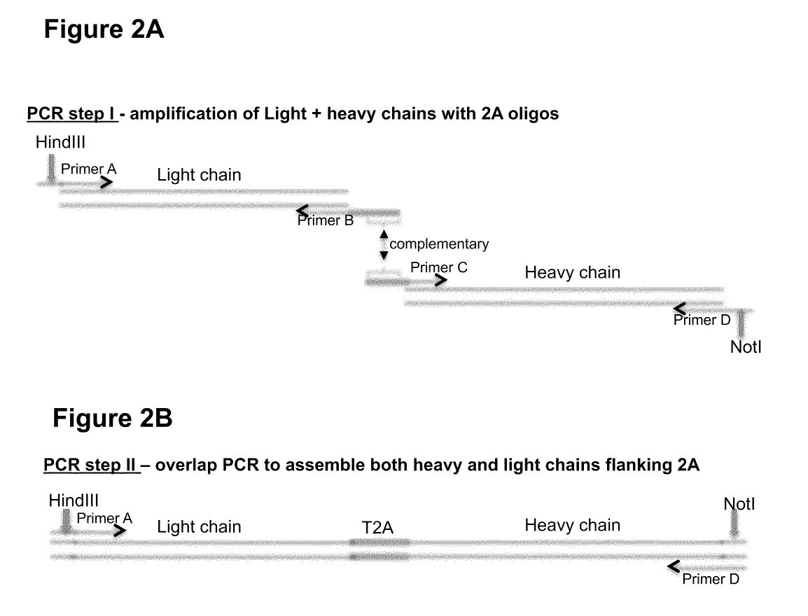

As described below in the Example 1 (see particularly FIGS. 2A and 2B), the nucleic acid cassette may be assembled prior to insertion of the entire cassette into a vector or the genome of an animal. However, it shall be understood that the components of a cassette of the invention may be inserted into the cassette in any order, and may be, in fact, inserted into the cassette after the cassette has been inserted into a vector. For example, as described below in Example 8, certain components of the cassette may be themselves assembled in a working vector, and the entire assembled cassette may then be moved from the working vector into an expression vector. Of course, some expression vectors, such as pcDNA3.1 (Invitrogen, Carlsbad, Calif.) are small enough to serve as both a working vector and the final expression vector containing the nucleic acid cassette.

Thus, used herein, by a “vector” is meant any construct capable of delivering one or more nucleic acid molecule(s) of interest to a host cell when the vector is introduced to the host cell or host animal. The host cell may be a eukaryotic or prokaryotic cell. In some embodiments, the cassette of the invention is constructed while components of the cassette are in a vector. In one non-limiting example, a vector comprising nucleic acid encoding the leader peptide sequence of a light chain of the antibody may be used as a backbone into which can be inserted a nucleic acid encoding the light chain of the antibody, the 2A peptide, the leader peptide of the heavy chain, and the heavy chain of the antibody.

An “expression vector” is capable of delivering and expressing the one or more nucleic acid molecule(s) of interest as encoded polypeptide in a host cell introduced with the expression vector. Thus, in an expression vector, a nucleic acid cassette is positioned for expression in the vector by being operably linked with regulatory elements such as a promoter, enhancer, polyA signal/tail, etc., either within the vector or in the genome of the host cell at or near or flanking the integration site of the nucleic acid molecule(s) of interest such that the nucleic acid molecule(s) of interest will be translated in the host cell introduced with the expression vector.

Additional, non-limiting regulatory elements to which a nucleic acid cassette of the invention may be operably linked to facilitate its expression when introduced into a cell include promoters (e.g., the phage lambda PL promoter, the E. coli lac, trp and tac promoters, the SV40 early and late promoters and promoters of retroviral LTRs), sites for transcription initiation, termination and, in the transcribed region, a ribosome binding site for translation. The coding portion of the mature transcripts expressed by an expression vector comprising a nucleic acid cassette may include a translation initiation codon at the beginning and a termination codon (UAA, UGA or UAG) appropriately positioned at the end of the nucleic acid cassette.

The nucleic acid cassette of the invention may be inserted into a vector containing a selectable marker for propagation in a host. In some embodiments, a plasmid vector is introduced in a precipitate, such as a calcium phosphate precipitate, or in a complex with a charged lipid. If the vector is a virus, it may be packaged in vitro using an appropriate packaging cell line and then transduced into host cells. The invention may be practiced with vectors comprising cis-acting control regions to the polynucleotide of interest. Appropriate trans-acting factors may be supplied by the host, supplied by a complementing vector or supplied by the vector itself upon introduction into the host. In certain embodiments in this regard, the vectors provide for specific expression, which may be inducible and/or cell type-specific (e.g., those inducible by environmental factors that are easy to manipulate, such as temperature and nutrient additives).

Thus, in another aspect, the invention provides a cell introduced with a nucleic acid cassette of the invention. In some embodiments, the cell expresses the nucleic acid cassette.

By “introduced” or “introducing” is meant that a nucleic acid molecule (e.g., a vector or a nucleic acid cassette) is inserted into the host cell by any means including, without limitation, electroporation, fusion with a vector-containing liposomes, chemical transfection (e.g., DEAE-dextran or calcium phosphate), transformation, cationic lipid-mediated transfection, transvection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents. Such methods are described in many standard laboratory manuals, such as Davis et al., BASIC METHODS IN MOLECULAR BIOLOGY (1986).

Any cell of any species may be introduced with the nucleic acid cassette of the invention. Thus, mammalian cells (e.g., HeLa cells, CV-1 cells, CHO cells), insect cells (e.g., Sf9 cells), yeast cells, and bacterial cells may be introduced with the nucleic acid cassette of the invention. In some embodiments, the introduced nucleic acid is positioned for expression in the cell such that the cell expresses the nucleic acid cassette (i.e., transcribes and/or translates the cassette) as antibody. To be expressed, the nucleic acid cassette may be on an expression vector, where the expression vector containing the nucleic acid cassette is introduced into a cell.

In some embodiments, the nucleic acid cassette of the invention may be introduced into a cell using a viral expression system (e.g., vaccinia or other pox virus, retrovirus, or adenovirus), which may involve the use of a non-pathogenic (defective), replication competent virus, or may use a replication defective virus. In the latter case, viral propagation generally will occur only in complementing virus packaging cells. Suitable systems are disclosed, for example, in Fisher-Hoch et al., 1989, Proc. Natl. Acad. Sci. USA 86:317-321; Flexner et al., 1989, Ann. N.Y. Acad. Sci. 569:86-103; Flexner et al., 1990, Vaccine 8:17-21; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner-Biotechniques 6:616-627, 1988; Rosenfeld et al., 1991, Science 252:431-434; Kolls et al., 1994, Proc. Natl. Acad. Sci. USA 91:215-219; Kass-Eisler et al., 1993, Proc. Natl. Acad. Sci. USA 90:11498-11502; Guzman et al., 1993, Circulation 88:2838-2848; and Guzman et al., 1993, Cir. Res. 73:1202-1207. Techniques for incorporating DNA into such expression systems are well known to those of ordinary skill in the art.

Of course, the inserted nucleic acid cassette of the invention need not be inserted into an expression vector prior to being introduced into a host cell. For example, a nucleic acid cassette may be introduced into a host cell by homologous recombination into the host cell's genome. If the inserted nucleic acid cassette is introduced into the genome such that it is operably linked to regulatory elements in that genome (e.g., the nucleic acid cassette is inserted downstream of a host cell's endogenous promoter), the nucleic acid cassette may be expressed (i.e., transcribed and translated) to allow the host cell to make recombinant antibody.

As indicated, when an expression vector is used, the expression vector may include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS and Bowes melanoma cells; and plant cells. Appropriate culture media and conditions for the above-described host cells are known in the art.

Non-limiting vectors for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Non-limiting eukaryotic vectors include pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Non-limiting bacterial promoters suitable for use in the present invention include the E. coli lad and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

In the yeast, Saccharomyces cerevisiae, a number of vectors containing constitutive or inducible promoters such as alpha factor, alcohol oxidase, and PGH may be used. For reviews, see Ausubel et al. (1989) CURRENT PROTOCOLS IN MOLECULAR BIOLOGY, John Wiley & Sons, New York, N.Y, and Grant et al., Methods Enzymol. 153: 516-544 (1997).

Transcription of DNA encoding an antibody of the present invention by higher eukaryotes may be increased by inserting an enhancer sequence into the vector. Enhancers are cis-acting elements of DNA, usually from about 10 to 300 by that act to increase transcriptional activity of a promoter in a given host cell-type. Examples of enhancers include the SV40 enhancer, which is located on the late side of the replication origin at basepairs 100 to 270, the cytomegalovirus early promoter enhancer, the polyoma enhancer on the late side of the replication origin, and adenovirus enhancers.

In some embodiments, the nucleic acid cassette of the invention (or an encoded antibody) may be purified. By “purified” (or “isolated”) refers to a molecule such as a nucleic acid sequence (e.g., a polynucleotide) or protein (e.g., an antibody) that is removed or separated from other components present in its natural environment. For example, an isolated antibody is one that is separated from other components of a eukaryotic cell (e.g., the endoplasmic reticulum or cytoplasmic proteins and RNA). An isolated antibody-encoding nucleic acid sequence (e.g., a sequence that is a component of a nucleic acid cassette of the invention) is one that is separated from other nuclear components (e.g., histones) and/or from upstream or downstream nucleic acid sequences (e.g., an isolated antibody-encoding polynucleotide may be separated from the endogenous heavy chain or light chain promoter). An isolated nucleic acid sequence or amino acid sequence of the invention may be at least 60% free, or at least 75% free, or at least 90% free, or at least 95% free from other components present in natural environment of the indicated nucleic acid sequence or amino acid sequence.

As used herein, the terms “peptide”, “polypeptide” and “protein” are used interchangeably herein to refer to polymers of amino acids of any length. The polymer may be linear or branched, and it may comprise modified amino acids. Where the amino acid sequence is provided, unless otherwise specified, the sequence is in an N′ terminal (amino-terminal) to C′ terminal (carboxy terminal) orientation (e.g., a PPL sequence is N′ proline-proline-leucine-C′). In some embodiments, the polymer may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon an antibody, the polypeptides can occur as single chains or associated chains.

In a further aspect, the invention provides a recombinant antibody produced by a cell expressing a nucleic acid cassette of the invention.

Naturally occurring antibodies are made up of two classes of polypeptide chains, light chains and heavy chains. A non-limiting antibody of the invention can be an intact, four immunoglobulin chain antibody comprising two heavy chains and two light chains. The heavy chain of the antibody can be of any isotype including IgM, IgG, IgE, IgA or IgD or sub-isotype including IgG1, IgG2, IgG2a, IgG2b, IgG3, IgG4, IgE1, IgE2, etc. The light chain can be a kappa light chain or a lambda light chain.

As used herein, the term “antibody” is meant to include an immunoglobulin molecule of any isotype (e.g., IgG1, IgG2a, IgG2b, IgG3, IgM, IgD, IgE, IgA) from any species (e.g., human, camelids (e.g., camels and llamas), chickens, goats, rabbits, and rodents (e.g., rats, mice, and hamsters). In some embodiments, an antibody encoded by a nucleic acid cassette of the invention specifically binds to a target molecule. As used herein, by “specifically binding” or “specifically binds” means that an antibody of the invention interacts with its target molecule, where the interaction is dependent upon the presence of a particular structure (i.e., the antigenic determinant or epitope) on the target molecule; in other words, the reagent is recognizing and binding to a specific structure rather than to all molecules in general. In some embodiments of the invention, an antibody that specifically binds to a target molecule provide a detection signal at least 5-, 10-, or 20-fold higher than a detection signal provided with other proteins when used in an immunochemical assay (e.g., (Western blotting, IHC, Immunofluorescence, etc.). In some embodiments, antibodies that specifically bind to a target molecule do not detect other proteins in immunochemical assays and can immunoprecipitate the target molecule from solution.

In some embodiments, an antibody encoded by a nucleic acid cassette of the invention has a K_{D} for its target molecule of 1×10⁻⁶M or less. In some embodiments, a binding agent of the invention binds to its target molecule with a K_{D} of 1×10⁻⁷ M or less, or a K_{D} of 1×10⁻⁸ M or less, or a K_{D} of 1×10⁻⁹ M or less, or a K_{D} of 1×10⁻¹° M or less, or a K_{D} of 1×10⁻¹¹M or less, or a K_{D} of 1×10⁻¹²M or less. In certain embodiments, the K_{D} of a binding agent of the invention for its target molecule is 1 μM to 500 μM, or between 500 μM to 1 μM, or between 1 μM to 100 nM, or between 100 mM to 10 nM. As used herein, by the term “K_{D}”, is intended to refer to the dissociation constant of an interaction between two molecules (e.g., the dissociation constant between a binding agent (e.g., an antibody) and its specific target molecule).

A single naturally-occurring antibody comprises two identical copies of a light chain and two identical copies of a heavy chain. The heavy chains, which each contain one variable domain (VH) and multiple constant region domains (CH1, hinge, CH2, and CH3), bind to one another via disulfide bonding within their constant domains to form the stem of the antibody. The light chains, which each contain one variable domain (VL) and one constant region domain (CL), each bind to one heavy chain via disulfide bonding. The variable domain of each light chain is aligned with the variable domain of the heavy chain to which it is bound. The variable regions of both the light chains and heavy chains contain three hypervariable regions known as the complementary determining regions (CDRs), sandwiched between four more conserved framework regions (FR) for a structure FR1, CDR1, FR2, CDR2, FR3, CDR3 and FR4.

As used herein, an “antigen binding domain” is any portion of an antibody that is capable of specifically binding to a target molecule and includes, without limitation, some or all of one or more of the following elements, CDR1, CDR2, CDR3, from either the heavy chain or the light chain.

Methods for identifying the CDR and FR regions of an antibody by analyzing the amino acid sequence of the antibody are well known (see, e.g., Wu, T. T. and Kabat, E. A. (1970) J. Exp. Med. 132: 211-250; Kabat, E. A. et al., Sequences of Proteins of Immunological Interest, National Institutes of Health, Bethesda, Md., (1987)); Martin et al., Methods Enzymol. 203:121-53 (1991); Morea et al., Biophys Chem. 68(1-3):9-16 (October 1997); Morea et al., J Mol. Biol. 275(2):269-94 (Jan. 1998); Chothia et al., Nature 342(6252):877-83 (December 1989); Ponomarenko and Bourne, BMC Structural Biology 7:64 (2007).

As one non-limiting example, the following method can be used to identify the CDRs of an antibody.

For the CDR-L1, the CDR-L1 is approximately 10-17 amino acid residues in length. Generally, the start is at approximately residue 24 (the residue before the 24^{th} residue is typically a cysteine. The CDR-L1 ends on the residue before a tryptophan residue. Typically, the sequence containing the tryptophan is either Trp-Tyr-Gln, Trp-Leu-Gln Trp-Phe-Gln, or Trp-Tyr-Leu, where the last residue within the CDR-L1 domain is the residue before the TRP in all of these sequences.

For the CDR-L2, the CDR-L2 is typically seven residues in length. Generally, the start of the CDR-L2 is approximately sixteen residues after the end of CDR-L1 and typically begins on the on the residue after the sequences of Ile-Tyr, Val-Tyr, Ile-Lys, or Ile-Phe.

For the CDR-L3, the CDR-L3 is typically 7-11 amino acid residues in length. Generally, the domain starts approximately 33 residues after the end of the CDR-L2 domain. The residue before the start of the domain is often a cysteine and the domain ends on the residue before Phe in the sequence Phe-Gly-XXX-Gly (where XXX is the three letter code of any single amino acid).

For the CDR-H1, the CDR-H1 domain is typically 10-12 amino acid residues in length and often starts on approximately residue 26. The domain typically starts four or five residues after a cysteine residue, and typically ends on the residue before a Trp (the Trp is often found in one of the following sequences: Trp-Val, Trp-Ile, or Trp-Ala. For the CDR-H2, the CDR-H2 domain is typically 16 to 19 residues in length and typically starts 15 residues after the final residue of the CDR-H1 domain. The domain typically ends on the amino acid residue before the sequence Lys/Arg-Leu/Ile/Val/Phe/Thr/Ala-Thr/Ser/Ile/Ala (which includes, for example, the sequences Lys-Leu-Thr and Arg-Ala-Ala).

For the CDR-H3, the CDR-H3 domain is typically 3-25 amino acids in length and typically starts 33 amino acid residues after the final residues of the CDR-H2 domain (which is frequently two amino acid residues after a cysteine residue, e.g., a cysteine in the sequence Cys-Ala-Arg). The domain ends on the amino acid immediately before the Trp in the sequence Trp-Gly-XXX-Gly (where XXX is the three letter code of any single amino acid; SEQ ID NO: 6)).

In a further aspect, the invention provides a nucleic acid cassette comprising components in the following structure: A-a-B-C-c, wherein “A” is a nucleic acid sequence encoding an antigen binding domain of a light chain of a first antibody, “a” is a nucleic acid sequence encoding a stem of a light chain of a second antibody, “B” is a nucleic acid sequence encoding a 2A peptide, “C” is a nucleic acid sequence encoding an antigen binding domain of a heavy chain of a third antibody, “c” is a nucleic acid sequence encoding a stem of a heavy chain of a fourth antibody, and “−” is a bond selected from the group consisting of a phosphodiester bond and a phosphorothioate bond. In some embodiments, the “−” is a phosphodiester bond.

As used herein, a “stem” is any portion of an antibody that is located, in the naturally occurring antibody, carboxy-terminally to the variable domain of the antibody and includes, without limitation, some or all of one or more of the following elements: a CH1 region, a hinge region, a CH2 region, a CH3 region, and a light chain constant region (CL region). An antibody encoded by a nucleic acid cassette of the invention may comprise a light chain constant region that comprises some or all of a CL region.

Antibodies encoded by the nucleic acid cassettes of the invention include but are not limited to polyclonal, monoclonal, monospecific, polyspecific antibodies and fragments thereof and chimeric antibodies comprising an immunoglobulin binding domain fused to another polypeptide. Similarly, antibodies encoded by the nucleic acid cassettes of the invention can be derived from any species of animal, including mammals (e.g., rabbit, mouse, human, rat). Non-limiting exemplary natural antibodies include antibodies derived from human, camelids (e.g., camels and llamas), chicken, goats, and rodents (e.g., rats, mice, hamsters and rabbits), including transgenic rodents genetically engineered to produce human antibodies (see, e.g., Lonberg et al., WO93/12227; U.S. Pat. No. 5,545,806; and Kucherlapati, et al., WO91/10741; U.S. Pat. No. 6,150,584, which are herein incorporated by reference in their entirety). Natural antibodies are the antibodies produced by a host animal. Genetically altered antibodies refer to antibodies wherein the amino acid sequence has been varied from that of a native antibody. Because of the relevance of recombinant DNA techniques to this application, one need not be confined to the sequences of amino acids found in natural antibodies; antibodies can be redesigned to obtain desired characteristics. The possible variations are many and range from the changing of just one or a few amino acids to the complete redesign of, for example, the variable or constant region. Changes in the constant region will, in general, be made in order to improve or alter characteristics, such as complement fixation, interaction with membranes and other effector functions. Changes in the variable region will be made in order to improve the antigen binding characteristics.

The antibody encoded by the nucleic acid cassette of the invention may be expressed in a modified form, such as a fusion protein (e.g., a GST-fusion), and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties may be added to the polypeptide to facilitate purification. Such regions may be removed prior to final preparation of the polypeptide. The addition of peptide moieties to polypeptides to engender secretion or excretion, to improve stability and to facilitate purification, among others, are familiar and routine techniques in the art.

In one non-limiting example, an antibody encoded by the nucleic acid cassette of the invention may comprise a heterologous region from an immunoglobulin that is useful to solubilize proteins. For example, EP-A-O 464 533 (Canadian counterpart 2045869) discloses fusion proteins comprising various portions of constant region of immunoglobulin molecules together with another human protein or part thereof. In many cases, the Fc part in a fusion protein is thoroughly advantageous for use in therapy and diagnosis and thus results, for example, in improved pharmacokinetic properties (EP-A 0232 262). On the other hand, for some uses it would be desirable to be able to delete the Fc part after the fusion protein has been expressed, detected and purified in the advantageous manner described. This is the case when Fc portion proves to be a hindrance to use in therapy and diagnosis, for example when the fusion protein is to be used as antigen for immunizations. In drug discovery, for example, human proteins, such as, hIL-5 has been fused with Fc portions for the purpose of high-throughput screening assays to identify antagonists of hIL-5. See Bennett et al., Journal of Molecular Recognition 8: 52-58 (1995) and Johanson et al., Journal of Biological Chemistry 270(16): 9459-9471 (1995).

In another aspect, the invention provides a method for making a recombinant antibody comprising introducing the nucleic acid cassette into a cell such that the nucleic acid cassette is expressed by the cell; maintaining the cell in a culture medium, and isolating the antibody from the cell or the culture medium.

The antibodies generated by using the nucleic acid cassettes of the invention can be recovered and purified from recombinant cell cultures by well-known methods including, without limitation, ammonium sulfate or ethanol precipitation, protein A-binding acid extraction, anion or cation exchange chromatography, phosphocellulose chromatography, hydrophobic interaction chromatography, affinity chromatography, hydroxylapatite chromatography and lectin chromatography. Where the antibody is not secreted, the host cell may first be lysed, and the antibody purified from the cell lysate. In some embodiments, high performance liquid chromatography (“HPLC”) is employed for purification. Depending upon the host employed in a recombinant production procedure (e.g., a eukaryotic or prokaryotic host cell), the antibodies generated using the nucleic acid cassettes of the present invention may be glycosylated or may be non-glycosylated. In addition, the antibodies may also include an initial modified methionine residue, in some cases as a result of host-mediated processes.

In some embodiments, the antibody generated as described herein are secreted by the host cell into which the nucleic acid cassette has been introduced. For secretion of the translated antibody into the lumen of the endoplasmic reticulum, into the periplasmic space or into the extracellular environment, appropriate leader peptides may be incorporated into the expressed polypeptide (or nucleic acid sequence encoding the same in the nucleic acid cassette).

Thus, in some embodiments, the nucleic acid cassette of the invention further comprises leader peptide sequences upstream (i.e., 5′) of the nucleic acid sequence encoding the light chain (or antigen binding domain thereof) and upstream of the nucleic acid sequence encoding the heavy chain (or antigen binding domain thereof). In some embodiments, the leader peptide sequence located 5′ to the nucleic acid sequence encoding the light chain (or antigen binding domain thereof) is a light chain leader peptide. In some embodiments, the leader peptide sequence located 5′ to the nucleic acid sequence encoding the heavy chain (or antigen binding domain thereof) is a heavy chain leader peptide.

As used herein, by “leader peptide” or a “secretory signal peptide” is meant a peptide sequence comprising a sequence that enables the polypeptide positioned C′ to the leader peptide to be secreted from a cell expressing (e.g., transcribing and/or translating) the polypeptide. In some embodiments, the leader peptide is attached to the polypeptide by a peptide bond. In some embodiments, the leader peptide may be cleaved from the polypeptide. In some embodiments, the cleavage of the leader peptide from the polypeptide occurs prior to the secretion of the polypeptide from the cell. The leader peptide may be endogenous to the polypeptide or it may be heterologous (i.e., the leader peptide naturally occurs N-terminally to a different molecule). Thus, leader peptide from a secreted hormone (e.g., cholecystokinin) is heterologous to light chain polypeptide.

Leader peptides (i.e., secretory signals) are well known in the art, since all secreted proteins (including antibodies) comprise them. For example, Barash et al., Biochemical and Biophysical Research Communications 294 (4): 835-842 (2002) describe a hidden Markov model (HMM) has been used to describe, predict, identify, and generate secretory signal peptide sequences. Similarly, U.S. Pat. No. 6,733,997 describes a universal secretory signal peptide sequence. A nucleic acid sequence encoding secretory signal peptide can be ligated, in frame, to a nucleic acid sequence encoding an antibody chain according to standard methods.

In further embodiments, the invention provides a method for producing a recombinant antibody by maintaining host cell comprising a nucleic acid cassette of the invention under conditions suitable for the expression of antibody and recovering the antibody, either from lysates made from the cells or, where the nucleic acid cassette included a secretory signal, from the conditioned media of the host cells. Culture conditions suitable for the maintenance and/or growth of host cells and the expression of recombinant polypeptides (such as antibodies) from such cells are well known to those of skill in the art. See, e.g., CURRENT PROTOCOLS IN MOLECULAR BIOLOGY, Ausubel F M et al., eds., Volume 2, Chapter 16, Wiley Interscience.

The invention also provides cell lines that produce an antibody encoded by the nucleic acid cassettes of the invention. For example, the invention includes recombinant host cells producing an antibody of the invention, which cells may be constructed by introducing into them the nucleic acid cassette of the invention. Host cells can be eukaryotic or prokaryotic cells (see, e.g., ANTIBODY ENGINEERING PROTOCOLS, 1995, Humana Press, Sudhir Paul editor.).

In some embodiments, it may be desirable to insert a nucleic acid sequence encoding a protease recognition site after the light chain-encoding nucleic acid sequence in the cassette. The addition of such a protease recognition site would enable cleavage of the 2A peptide from the light chain encoded by the nucleic acid cassette of the invention. Accordingly, in another aspect, the invention provides a nucleic acid cassette comprising components in the following structure:

A-p-B-C, where “A” is a nucleic acid sequence encoding at least an antigen binding domain of a light chain of a first antibody, “B” is a nucleic acid sequence encoding a 2A peptide, “C” is a nucleic acid sequence encoding at least an antigen binding domain of a heavy chain of a second antibody, “−” is a phosphodiester bond and a phosphorothioate bond, and “p” “p” is nucleic acid sequence encoding a protease recognition site.

As used herein, by “protease recognition site” is meant a specific amino acid sequence within a polypeptide (e.g., a protein) at which or after which a protease will cleave the polypeptide. Such proteases and their recognition sites include, without limitation, the furin protease (which cleaves after the sequence the final arginine residue in the sequences Arg-X-X-Arg (SEQ ID NO: 7); Arg-X-Lys-Arg (SEQ ID NO: 8); or Arg-X-Arg-Arg (SEQ ID NO: 9), where X is any amino acid residue), the enterokinase protease (which cleaves after the final lysine residue in the sequence Asp-Asp-Asp-Asp-Lys; SEQ ID NO: 10), and the Factor Xa protease (which cleaves after the final arginine residues in the sequences Ile-Glu-Gly-Arg (SEQ ID NO: 11) or Ile-Asp-Gly-Arg (SEQ ID NO: 12)). All of the former proteases are commercially available (e.g., from New England Biolabs, Inc.).

The antibodies generated in accordance with the present invention may be employed in various methods. For example, the antibodies of the invention may be used in any known assay method, such competitive binding assays, direct and indirect sandwich assays, and immunoprecipitation assays. Zola, Monoclonal Antibodies: A Manual of Techniques, pp. 147-158 (CRC Press, Inc. 1987). For use in in vitro assays, the antibodies may be detectably labeled (e.g., with a fluorophore such as FITC or phycoerythrin or with an enzyme substrate, such as a substrate for horse radish peroxidase) for easy detection. The antibodies may also be generated such that one or both of the heavy and light chain is tagged.

Accordingly, in another aspect, the invention provides a nucleic acid cassette comprising components in the following structure:

A-B-C-D or A-D-B-C,

where “A” is a nucleic acid sequence encoding at least an antigen binding domain of a light chain of a first antibody, “B” is a nucleic acid sequence encoding a 2A peptide, “C” is a nucleic acid sequence encoding at least an antigen binding domain of a heavy chain of a second antibody, “−” is a phosphodiester bond and a phosphorothioate bond, and “D” is a nucleic acid sequence encoding a tag,

As used herein, a “tag” means a peptide structure that can be detected. Thus, a tag includes, without limitation, an epitope that can be recognized by an antibody, the ligand of a receptor, one partner of a binding partner pair (e.g., the streptavidin-biotin binding partner pair), a mass tag that produces an identifiable spectrum by mass spectrometry, a fluorescent label, a chromophoric label, and a marker protein. By “marker protein” is meant a polypeptide whose expression or activity indicates the amount of the polypeptide in the sample (e.g., in a cell). Non-limiting marker proteins include green fluorescent protein and horseradish peroxidase.

Non-limiting examples of tags (and their amino acid sequences) include the c-myc tag (EQKLISEEDL; SEQ ID NO: 13), the His tag (HHHHHH; SEQ ID NO: 14); the HA tag (YPYDVPDYA; SEQ ID NO: 15), the VSV-G tag (YTDIEMNRLGK; SEQ ID NO: 16), the HSV tag (QPELAPEDPED; SEQ ID NO: 17), the V5 tag (GKPIPNPLLGLDST; SEQ ID NO: 18), and the Flag tag (DYKDDDDK; SEQ ID NO: 19).

In some embodiments, the tag identifies the source of the antibody encoded by the nucleic acid cassette.

In some embodiments, the tag is cleavable from the chain of the antibody. For example, as described below, many proteases (e.g., furin, Factor Xa, etc.) cleave at specific sites. Thus, a nucleic acid sequence encoding a protease recognition site (e.g., the Factor Xa protease recognition site Ile-Glu-Gly-Arg (SEQ ID NO: 11) or Ile-Asp-Gly-Arg (SEQ ID NO: 12)), can be inserted between the nucleic acid sequence encoding the heavy chain of an antibody and the nucleic acid sequence encoding the tag. This configuration would result in a nucleic acid cassette comprising components in the following structure:

A-B-C-p-D or A-p-D-B-C,

where “A” is a nucleic acid sequence encoding at least an antigen binding domain of a light chain of a first antibody, “B” is a nucleic acid sequence encoding a 2A peptide, “C” is a nucleic acid sequence encoding at least an antigen binding domain of a heavy chain of a second antibody, “−” is a phosphodiester bond and a phosphorothioate bond, “D” is a nucleic acid sequence encoding a tag, and “p” is a nucleic acid sequence encoding a protease recognition site.

Note that additional elements can be inserted in component D in the above-described nucleic acid cassette. Thus, instead of a tag, the D can be nucleic acid sequence encoding any polypeptide. Some non-limiting examples include the J chain of an antibody (e.g., where an IgM isotype antibody is encoded by components A and C) or a selectable marker. In the latter example, where D encodes a selectable marker (e.g., neomycin resistance), cells expressing the nucleic acid cassette will be able to survive and/or grow in the presence of the drug (e.g., G418).

In further embodiments the antibodies generated in accordance with the invention may be used for in vivo diagnostic assays, such as in vivo imaging. In some embodiments, the antibody is labeled with a radionucleotide (such as ³H, ¹¹¹In, ¹⁴C, ³²P, or ¹²³I) so that the cells or tissue of interest can be localized using immunoscintigraphy.

Methods of conjugating labels or tags to antibodies are known in the art. In other embodiments of the invention, the antibodies generated in accordance with the invention are not labeled, and the presence thereof is detected using a labeled secondary antibody, which binds to the first, unlabeled antibody.

The antibody may also be used as staining reagent in pathology, following techniques well known in the art.

In another aspect, the invention provides a kit comprising a first primer comprising a 5′ portion comprising a recognition site of a first restriction endonuclease and a 3′ portion that hybridizes to an antisense strand of a nucleic acid sequence encoding a leader peptide of a light chain of a first antibody; a second primer comprising a 5′ portion comprising a nucleic acid sequence that is complementary a nucleic acid sequence that encodes a first part of a 2A peptide and a 3′ portion that hybridizes to a nucleic acid sequence encoding a constant region of a light chain of a second antibody; a third primer comprising a 5′ portion comprising a nucleic acid sequence that encodes a second part of a 2A peptide and a 3′ portion that hybridizes to an antisense strand of a nucleic acid sequence encoding a leader peptide of a heavy chain of a third antibody; a fourth primer comprising a 5′ portion comprising a recognition site of a second restriction endonuclease and a 3′ portion that hybridizes to a nucleic acid sequence encoding a constant region of a heavy chain of a fourth antibody; and instructions for using the first, second, third, and fourth primers to generate a nucleic acid cassette from a sample comprising nucleic acid encoding the first antibody, the second antibody, the third antibody, and the fourth antibody.

As used herein, by “hybridize” is meant that a primer (e.g., a PCR primer) anneals to another single stranded nucleic acid molecule to form a double stranded nucleic acid molecule. In general, hybridization (i.e., annealing) should occur under stringent conditions.

A typical PCR program consists of:
Step 1—95 C for 2-5 minutes
Step 2—95 C for 30 seconds
Step 3—55-72 C for 30 seconds
Step 4—72 C for 1 minute/kb of product
Step 5—go back to Step 2 29 times (for 30 cycle reaction)—can be adjusted as needed
Step 6—72 C for 5-10 minutes

The annealing temperature at Step 3 can be raised to increase specificity of priming (i.e., increasing stringency). Typically, it is set at 2-5 degrees lower than the predicted Tm of the oligos (which should have similar Tm to each others'), but it can be raised to as high as the extension temperature (Step 4). Another method for increasing the specificity of priming (i.e., increasing stringency) is by decreasing Mg2+ concentration to below 2 mM. To determine Tm of a primer, any of a variety of methods may be used.

One non-limiting formula is:

Tₘ=81.5°C.+16.6(log [M+])+0.41(% G+C)−(500/N), where [M+] is the concentration of monovalent cation in the PCR reaction (e.g., KCl) in moles/liter, N is the number of nucleotides in the primer, and % G+C is the percentage of G and C residues in the primer (e.g., a 14 nucleotide long primer with 4 G residues and 3 C residues has a % G+C of 50%).

Another non-limiting method for determining Tm of a primer is:

Tₘ=64.9° C.+41° C.×(number of G's and C's in the primer−16.4)/N, where N is the number of nucleotides in the primer.

Thus, as used herein, by “stringent conditions” is meant that the primer will hybridize (i.e., anneal) to the single stranded target molecule (a) within a range from about Tₘ minus 2° C. (2° C. below the melting temperature (Tₘ) of the probe or sequence) to about 20° C. to 25° C. above Tₘ or (b) in a solution containing a concentration of Mg2+ that is equal to or less concentrated than 2 mM. It will be understood by those of skill in the art that the stringency of hybridization may be determined on a PCR.

As used herein, by “portion” is meant any subset of the whole sequence of the primer. Thus, a portion of a primer of 20 nucleotides in length is meant any sequence that is 19 nucleotides in length or fewer. Of course, if there are two portions in the same primer (e.g., a 5′ portion and a 3′ portion), one of the two portions is at least 25% as long as the other portion. For example, in the 20 nucleotide long primer, the 5′ portion may comprise 5 nucleotides or more and the 3′ portion may comprise 15 nucleotides or fewer. In some embodiments, where there are two portions in the same primer, one of the two portions is at least 30% or at least 35% or at least 40% as long as the other portion.

In some embodiments of the kit of the invention, the first antibody and the second antibody are the same. In some embodiments, the third antibody and the fourth antibody are the same. In some embodiments, the first antibody, second antibody, third antibody, and fourth antibody are the same.

In some embodiments, the kit further comprises a thermostable DNA polymerase (e.g., Taq polymerase). In some embodiments, the kit further comprises a first restriction endonuclease and a second restriction endonuclease. In some embodiments, the first and the second restriction endonuclease are the same. For example, where a restriction endonuclease recognizes a site that has a degenerate (i.e., variable) sequences, the same restriction endonuclease can recognize two different recognition sites. For example, the restriction endonuclease SfiI recognizes the site: 5′GGCCNNNN*NGGCC3′ (SEQ ID NO: 20) cutting at the * to create a 3′ overhang of —NNNN 3′, where “N” can be any nucleotide. Those “N” sequences in the first and second endonuclease recognition sites can differ, but both the first and the second endonuclease sites would still be recognized and cut by SfiI.

In some embodiments, the kit further comprises a vector comprising a polylinker comprising the first restriction endonuclease recognition site and the second restriction endonuclease recognition site. In some embodiments, the kit further comprises a vector fragment of a vector comprising a polylinker comprising the first restriction endonuclease recognition site and the second restriction endonuclease recognition site digested with the first restriction endonuclease and the second restriction endonuclease.

As used herein, by “polylinker” is meant a portion of a vector (e.g., an expression vector) that contains many unique restriction endonuclease recognition sites (i.e., the site does not occur elsewhere in the vector). Typically, in an expression cloning vector, the polylinker occurs downstream of a promoter sequence and upstream of a polyA signal.

In a further aspect, the invention provides a method for making a nucleic acid cassette. The method comprises (a) amplifying a nucleic acid molecule encoding a light chain comprising a leader peptide and a constant region of a first antibody with a first primer comprising a 5′ portion comprising a recognition site of a first restriction endonuclease and a 3′ portion that hybridizes to an antisense strand of a nucleic acid sequence encoding the leader peptide of the light chain and a second primer comprising a 5′ portion comprising a nucleic acid sequence that is complementary to a first part of a 2A-peptide encoding nucleic acid sequence and a 3′ portion that hybridizes to a nucleic acid sequence encoding the constant region of the light chain and (b) amplifying a nucleic acid molecule encoding a heavy chain comprising a leader peptide and a constant region of a second antibody with a third primer comprising a 5′ portion comprising a nucleic acid sequence that encodes a second part of a 2A peptide and a 3′ portion that hybridizes to an antisense strand of a nucleic acid sequence encoding the leader peptide of the heavy chain and a fourth primer comprising a 5′ portion comprising a recognition site of a second restriction endonuclease and a 3′ portion that hybridizes to a nucleic acid sequence encoding the constant region of the heavy chain of a third antibody. In step (c), the products of step (a) and step (b) are allowed to anneal (i.e., hybridize) and in step (d), the product of step (c) is amplified with the first primer and the fourth primer. In some embodiments, the amplifying step is by polymerase chain reaction (PCR) amplification.

The following examples are provided to illustrate, but not to limit, the invention.

### Example 1

### Construction of a Light Chain-2A-Heavy Chain Nucleic Acid Cassette Encoding an Anti-MRPL11 Rabbit IgG

The light chain-2A-heavy chain (L-2A-H) cassette encoding an anti-MRPL11 rabbit IgG antibody was assembled as a single molecule of DNA using two steps of polymerase chain reaction (PCR). This two step process is schematically depicted in FIGS. 2A and 2B, respectively. Briefly, the first step consisted of two independent reactions, one that amplified the light chain (or 5′ piece of the cassette) with Primer A and Primer B from a template encoding the full length light chain sequence including the light chain leader sequence, and the other that amplifies the heavy chain (or 3′ end of the cassette) with Primer C and Primer D from a template encoding the full length heavy chain sequence including the heavy chain leader sequence. The sequences of Primers A-D were as follows (where the sequences derived from rabbit sequences are underlined):

(SEQ ID NO: 21)

Primer A: 5′ GTCGTCAAGCTTGACATGGACATGAGGGCCCCC 3′

(SEQ ID NO: 22)

Primer B: 5′
CTCCACGTCACCGCATGTTAGAAGACTTCCTCTGCCCTCACAGTCACCC
CTATTGAAGCT 3′

(SEQ ID NO: 23)

Primer C: 5′
TCTTCTAACATGCGGTGACGTGGAGGAGAATCCCGGCCCTATGGAGAC
TGGGCTGCGCT 3′

(SEQ ID NO: 24)

Primer D: 5′ ATAAGAATGCGGCCGCTATCATTTACCCGGAGAG
CGGGA 3′

Primer A was designed to hybridize to the 5′ end of the rabbit kappa chain leader sequence and contains additional 5′ sequence that includes a HindIII restriction site. Primer B was designed to hybridize to the 3′ end of the rabbit kappa chain constant region and also contains 39 nucleotides encoding the N-terminal half of the T2A peptide sequence in its 5′ end. Primer C was designed to hybridize to the 5′ end of the rabbit heavy chain leader sequence and also contains 40 nucleotides encoding the C-terminal half of the T2A peptide sequence in its 5′ end. Note that the number of nucleotides encoding the T2A peptide (or any other 2A peptide used) in Primers B and C may be varied as long as they contain enough complementary nucleotides (about 20 nucleotides) to each other for efficient assembly in the second step of PCR.

Primer D was designed to hybridize to the 3′ end of sequence encoding the rabbit heavy chain constant region 3 and contained additional 5′ sequence that includes a Not I restriction site.

In this example, Primer B and Primer C are complementary in their 5′ portions, allowing hybridization to generate a template for the full length cassette in the second PCR step, when Primer A and Primer D are used to assemble the light chain and heavy chain sequences flanking the T2A peptide sequence. This method of using 4 primers to generate the final full length cassette is a modification of a method called an overlap PCR (or overlap extension PCR) (see Higuchi et al., Nucleic Acids Res. 16(15): 7351-67 (1988). The sequence of the T2A peptide (i.e., the 2A peptide from Thosea asigna) comprises the amino acid sequence N-terminus-EGRGSLLTCGDVEENPGP —C-terminus, where the ribosomal skip (i.e., no peptide bond is formed) between the C-terminal GP (underlined and bolded in the above sequence). These four primers may not be restricted to rabbit IgG sequence but can be designed to amplify IgG of any species including but not limited to human, mouse and chicken. Note that in the case of human or mouse IgG (or any other species where a diverse repertoire of IgG sequences exist), the primer pairs used for the amplification and assembly of the light and heavy chains may contain a set of multiple primers that hybridize to a range of sequences. In addition, the recognition sites in Primer A and Primer D are not limited to HindIII and NotI restriction enzymes for ligation. As mentioned above, a single restriction endonuclease with an interrupted palindromic recognition site with degenerate sequence (such as SfiI, AleI, BstAPI, DraIII etc.) may also be used instead of two distinct enzymes.

In all cases, 3′ end sequences of Primers B and C contain complementary 2A peptide-encoding sequences that are part of the nucleic acid cassette of the invention. The 2A peptide sequence in Primers B and C may not be restricted to T2A (from Thosea asigna virus) but may be designed from 2A peptides of any of the Aphthoviruses.

For the first step of the amplification, the following PCR protocol was used:

Step 1—95 C for 5 minutes
Step 2—95 C for 30 seconds
Step 3—55 C for 30 seconds
Step 4—72 C for 1 minute
Step 5—go back to Step 2, 29 times
Step 6—72 C for 5 minutes

Amplification of the light chain with Primer A and Primer B generated a DNA fragment of approximately 750 bp, and amplification of the heavy chain with Primer C and Primer D generated a DNA fragment of approximately 1.4 kbp. DNA fragments generated in each reaction in the first PCR step was visualized in an ethidium bromide-stained 0.7% TAE agarose gel and gel-purified. 1/30^{th} of each purified DNA fragment eluate after gel purification was mixed in a subsequent reaction for the second PCR step with Primer A and Primer D to assemble and amplify the full-length L-2A-H cassette. The second PCR step used the following PCR protocol:

Step 1—95 C for 5 minutes
Step 2—95 C for 30 seconds
Step 3—55 C for 30 seconds
Step 4—72 C for 2 minutes
Step 5—go back to Step 2, 29 times
Step 6—72 C for 5 minutes

In this manner, the following distinct L-2A-H cassette (nucleic acid sequence in a 5′ to 3′ orientation and amino acid sequence in a N-terminus to C-terminus orientation) were generated. (The cassette was sequenced according to standard methods.) Note that the below sequence includes only the coding region, and therefore does not show the HindIII restriction endoncuclease recognition site at the 5′ end or the NotI restriction endoncuclease recognition site at the 3′ end.

MRPL11 IgG L-2A-H cassette (nucleotide sequence)
(SEQ ID NO: 25)
ATGGACATGAGGGCCCCCACTCAGCTGCTGGGGCTCCTGCTGCTCTGGCTCCC
AGGTGCCACATTTGCCCAAGTGCTGACCCAGACTCCATCCCCCGTGTCTGCAG
CTGTGGGAAACACAGTCACCATCAATTGCCAGGCCAGTCAGAGTGTTCGTGA
TAATAACTACTTATCCTGGTATCAGCAGAAACCAGGTCAGCCTCCCAAGCTC
CTGATCTACAGGGCATCCACTCTGGAATCTGGGGTCCCATCGCGGTTTAAAG
GCAATGGATCTGGGACACAATTCACTCTCACCATCAGCGACCTGGAGTGTGA
CGATGCTGCCACTTACTATTGTCAAGGCGGTTATGGTGGGAATTTTTTTCCTTT
CGGCGGAGGGACCGAGGTGGTGGTCAAAGGTGATCCAGTTGCACCTACTGTC
CTCATCTTCCCACCAGCTGCTGATCAGGTGGCAACTGGAACAGTCACCATCGT
GTGTGTGGCGAATAAATACTTTCCCGATGTCACCGTCACCTGGGAGGTGGAT
GGCACCACCCAAACAACTGGCATCGAGAACAGTAAAACACCGCAGAATTCT
GCAGATTGTACCTACAACCTCAGCAGCACTCTGACACTGACCAGCACACAGT
ACAACAGCCACAAAGAGTACACCTGCAAGGTGACCCAGGGCACGACCTCAG
TCGTCCAGAGCTTCAATAGGGGTGACTGTGAGGGCAGAGGAAGTCTTCTAAC
ATGCGGTGACGTGGAGGAGAATCCCGGCCCTATGGAGACTGGGCTGCGCTGG
CTTCTCCTGGTCGCTGTGCTCAAAGGTGTCCAGTGTCAGTCGGTGGAGGAGTC
CGGGGGTCGCCTGGTCAAGCCTGACGAAACCCTGACAATCACCTGCACAGTC
TCTGGAATCGACCTCAATAACAATGCAATGGGCTGGGTCCGCCAGGCTCCAG
GGGAGGGGCTGGAATACATCGGATTCATTGGTGGGAGTGGTGCCACATACTA
CTCGACCTGGGCGAAAGGCCGGTTCACCATCTCCAAGTCCTCGACCACGGTG
GATCTGATGATCACCAGTCCGACAACCGAGGACACGGCCACCTATTTCTGTG
CCAGATATGCTGGTAGTGGTTCTTTTGACTTCTCGGGCCCAGGCACCCTGGTC
ACCGTCTCCTTGGGGCAACCTAAGGCTCCATCAGTCTTCCCACTGGCCCCCTG
CTGCGGGGACACACCCAGCTCCACGGTGACCCTGGGCTGCCTGGTCAAAGGC
TACCTCCCGGAGCCAGTGACCGTGACCTGGAACTCGGGCACCCTCACCAATG
GGGTACGCACCTTCCCGTCCGTCCGGCAGTCCTCAGGCCTCTACTCGCTGAGC
AGCGTGGTGAGCGTGACCTCAAGCAGCCAGCCCGTCACCTGCAACGTGGCCC
ACCCAGCCACCAACACCAAAGTGGACAAGACCGTTGCGCCCTCGACATGCAG
CAAGCCCACGTGCCCACCCCCTGAACTCCTGGGGGGACCGTCTGTCTTCATTT
TCCCCCCAAAACCCAAGGACACCCTCATGATCTCACGCACCCCCGAGGTCAC
ATGCGTGGTGGTGGACGTGAGCCAGGATGACCCCGAGGTGCAGTTCACATGG
TACATAAACAACGAGCAGGTGCGCACCGCCCGGCCGCCGCTACGGGAGCAG
CAGTTCAACAGCACGATCCGCGTGGTCAGCACCCTCCCCATCGCGCACCAGG
ACTGGCTGAGGGGCAAGGAGTTCAAGTGCAAAGTCCACAACAAGGCACTCC
CGGCCCCCATCGAGAAAACCATCTCCAAAGCCAGAGGGCAGCCCCTGGAGCC
GAAGGTCTACACCATGGGCCCTCCCCGGGAGGAGCTGAGCAGCAGGTCGGTC
AGCCTGACCTGCATGATCAACGGCTTCTACCCTTCCGACATCTCGGTGGAGTG
GGAGAAGAACGGGAAGGCAGAGGACAACTACAAGACCACGCCGGCCGTGCT
GGACAGCGACGGCTCCTACTTCCTCTACAGCAAGCTCTCAGTGCCCACGAGT
GAGTGGCAGCGGGGCGACGTCTTCACCTGCTCCGTGATGCACGAGGCCTTGC
ACAACCACTACACGCAGAAGTCCATCTCCCGCTCTCCGGGTAAATGA
MRPL11 IgG L-2A-H cassette (amino acid sequence)
(SEQ ID NO: 26)
M D M R A P T Q L L G L L L L W L P G A T F A\hat{ }Q V L T Q T P S P V S A A V G N
T V T I N C Q A S Q S V R D N N Y L S W Y Q Q K P G Q P P K L L I Y R A S T L E
S G V P S R F K G N G S G T Q F T L T I S D L E C D D A A T Y Y C Q G G Y G G
N F F P F G G G T E V V V K(G D P V A P T V L I F P P A A D Q V A T G T V T I V
C V A N K Y F P D V T V T W E V D G T T Q T T G I E N S K T P Q N S A D C T Y
N L S S T L T L T S T Q Y N S H K E Y T C K V T Q G T T S V V Q S F N R G D C)
E G R G S L L T C G D V E E N P G_P M E T G L R W L L L V A V L K G V Q
C\hat{ }Q S V E E S G G R L V K P D E T L T I T C T V S G I D L N N N A M G W V R Q
A P G E G L E Y I G F I G G S G A T Y Y S T W A K G R F T I S K S S T T V D L M
I T S P T T E D T A T Y F C A R Y A G S G S F D F S G P G T L V T V S L(G Q P K
A P S V F P L A P C C G D T P S S T V T L G C L V K G Y L P E P V T V T W N S G
T L T N G V R T F P S V R Q S S G L Y S L S S V V S V T S S S Q P V T C N V A H
P A T N T K V D K T V A P S T C S K P T C P P P E L L G G P S V F I F P P K P K D
T L M I S R T P E V T C V V V D V S Q D D P E V Q F T W Y I N N E Q V R T A R
P P L R E Q Q F N S T I R V V S T L P I A H Q D W L R G K E F K C K V H N K A L
P A P I E K T I S K A R G Q P L E P K V Y T M G P P R E E L S S R S V S L T C M I
N G F Y P S D I S V E W E K N G K A E D N Y K T T P A V L D S D G S Y F L Y S
K L S V P T S E W Q R G D V F T C S V M H E A L H N H Y T Q K S I S R S P G K)

Note that in the above amino acid sequence, the predicted leader cleavage sites are indicated with a “̂” symbol, the CDRs are all underlined, the constant region is placed in parentheses, and the T2A sequence is bolded (where the “_” symbol indicates the translational skip within the T2A sequence).

### Example 2

### Generation of Additional L-2A-H Nucleic Acid Cassettes

Using the same methods and same primers set forth in Example 1, an additional three cassettes were generated, namely a ERK2p IgG L-2A-H cassette (i.e., encoding an anti-ERK2p rabbit IgG antibody), a SUZ12 IgG L-2A-H cassette (i.e., encoding an anti-SUZ12 rabbit IgG antibody), and HER2 IgG L-2A-H cassette (i.e., encoding an anti-HER2 rabbit IgG antibody).

### Example 3

### Insertion of the L-2A-H Nucleic Acid Cassettes into a Replicable Plasmid Vector

To subclone the L-2A-H nucleic acid cassettes into plasmid vectors, the approximately 2 kb products from the second step PCR were gel purified and digested with HindIII and NotI (both from New England Biolabs) to generate directionally ligatable 5′ and 3′ ends. These fragments with “sticky ends” were then ligated using T4 DNA ligase (New England Biolabs, Ipswich, Mass.) into the vector fragment of either the pTT5 mammalian expression vector (from the National Research Council Canada) or the pCDNA3 expression vector (from Invitrogen, Carlsbad, Calif.) digested with HindIII and NotI. Note that expression vector need not be limited to this vector and any other vector (e.g., a eukaryotic expression vector such as pCI-Neo or simply a cloning vector such as puc9) may be applicable.

Competent E. coli were transformed with the ligation reactions and selected on LB ampicillin agar plates, and single colonies were inoculated into LB ampicillin broth for overnight growth. Plasmid DNA was isolated from the liquid cultures using a commercially available kit (Zymo Research), and the presence of the L-2A-H cassette insert in the plasmids was verified by visualization of a 2 kbp fragment on a 0.7% TAE gel following a HindIII/NotI digest.

### Example 4

### Expression of IgG in Mammalian Cells from Expression Plasmids

The L-2A-H cassettes inserted into either the pTT5 or the pcDNA3 expression vectors were next transfected into mammalian cells. In addition, a heavy chain-2A-light chain (H-2A-L cassette) inserted into pTT5 or pCDNA3 mammalian expression vector was also transfected into mammalian cells. As a negative control, pTT5 vector or pCDNA3 vector with no insert was transfected into mammalian cells, while as a positive control, mammalian cells were transfected with two separate vectors (either pTT5 or pcDNA3, depending upon which vector backbone was used for the nucleic acid cassette), one encoding the light chain and one encoding the heavy chain.

To do this, each expression vector (or pair of expression vectors for H+L) was transiently transfected into HEK293T cells (commercially available from the American Type Culture Collection, Manassas, Va.) plated at approximately 80% confluency on 12-well plates in 1 ml/well of Dulbecco's Modified Eagle Medium (DMEM) supplemented with 10% fetal bovine serum, 100 IU/ml penicillin, 100 μg/ml streptomycin and 2 mM L-glutamine and incubated at 37° C. with 5% CO₂. Note that the cells used for transfection need not be limited to HEK293T cells, but can be any other transfectable cell line, and can be transfected for transient or stable expression. For the transfection, the DNA to be transfected was complexed with a transfection carrier as follows. 1 μg of DNA was diluted in 25 μl of serum-free DMEM, 100 μl of serum-free DMEM containing 74 μg/ml of polyethylenimine was added to the diluted DNA and mixed gently, incubated at room temperature for 30 minutes, then gently added onto HEK293T cells that were seeded 24 hours prior. 5 days later, the supernatant was harvested for characterization of secreted IgG, and the cells were lysed in 100 μl of 1×Laemmli buffer (with 42 mM DTT) for Western blot analyses. As controls for each antibody, 1 μg of a 1:1 mixture of heavy chain- and light chain-encoding pTT5 plasmids or pcDNA3 plasmids were transfected in the same exact manner. Expression of IgG was driven by a CMV immediate early promoter in the vectors tested (i.e., pTT5 or pcDNA3), but other eukaryotic promoters (e.g., the spleen focus-forming virus (SFFV) promoter or the EF1alpha promoter) may also be used. Although transfection here was by chemical means, physical transfection (e.g., electroporation) may also be used. In fact, any method for inserting the expression vectors containing the nucleic acid cassette into a cell line may be used (e.g., transduction, infection, etc. . . . ).

### Example 5

### Characterization of Secreted IgG by ELISA

The culture supernatant from the transfected cells harvested 5 days post-transfection was characterized for secretion of IgG with specific binding activity to target antigens by enzyme-linked immunosorbent assay (ELISA). High-binding 96-well polystyrene plates (Costar) were coated with 0.1 μg of antigen (immunizing peptides for HER2, ERK2p, MRPL11 or SUZ12 in the case of target antigens, or anti-rabbit IgG antibody for detection of total IgG) and blocked with 5% bovine serum albumin in tris-buffered saline (TBS). Each supernatant sample was tested at undiluted and diluted 10-fold in TBS when tested against peptides and diluted 100- and 1000-fold when tested for total IgG on plates coated with anti-rabbit IgG antibody. 50 μl of each supernatant was added per well and plates were incubated at 37° C. for 2 hours, after which the plates were washed 3 times with TBS-Tween (0.1%) (TBS-T), 50 μl of detection antibody (anti-rabbit HRP, Cell Signaling Technology, Inc., Danvers, Mass., product #7074) diluted 2000-fold in TBS-T was added to each well and plates were incubated at 37° C. for 1 hour then washed 3 times, and finally developed with 50 μl of TMB solution (BioFX labs), neutralized with 50 μl of stop solution (BioFX labs), and OD450 nm was read on a plate reader (Titertek). Tables 1-4 show the absorbance values of ELISAs for anti-ERK2p, anti-MRPL11, anti-SUZ12, and anti-HER2 antibodies, respectively. Each IgG was constructed and tested in both the L-2A-H and H-2A-L configurations. All samples tested were generated from a single transfection experiment. Total IgG concentration in each sample was quantified in a separate ELISA by comparing the signal to a standard curve ranging from 2 ng/ml to 0.05 ng/ml.

In each table, “sup dilution” means the factor by which the culture supernatant was diluted in the ELISA assay, “vector only” means supernatant taken from cells transfected with empty pTT5 vector or empty pcDNA3, and “H+L+ve” means supernatant taken from cells transfected with two vectors, one containing the H chain and one containing the L chain. For each configuration (L-2A-H and H-2A-L) of the antibody, with the exception of the anti-ERK2p L-2A-H configuration, supernatants from two independent clones (indicated in Table 1 as H2AL#10 and #19, for example) were tested to demonstrate reproducibility.

Tables 1, 2, 3, and 4 respectively, show the binding specificity and quantification of anti-ERK2p, anti-MRPL11, anti-SUZ12, and anti-HER2 rabbit IgG. Supernatants of HEK293T cells transfected with each antibody cassette vector was tested for binding to all three antigens by ELISA at undiluted and 10-fold dilution. Total IgG secretion (bottom row, [IgG] ug/ml) was tested qualitatively (at 1/100 and 1/1000 dilutions) and quantitatively by quantitative ELISA-determined concentration. For samples where the IgG concentration is not indicated, the concentrations were not measured. The numbers in the table are absorbance values of optical density measured at 450 nm and are averages of duplicate measurements.

TABLE 1
Erk2p Antibody

| | sup | vector | H2AL | H2AL | L2AH | |
|---|---|---|---|---|---|---|
| Antigen | dilution | only | #10 | #19 | #2 | H + L + ve |
| Erk2P | 1× | 0.05035 | 1.15775 | 1.07505 | 1.03765 | 0.9168 |
| | 1/10× | 0.05075 | 0.89115 | 1.036 | 1.0942 | 0.9732 |
| MRPL11 | 1× | 0.04585 | 0.0628 | 0.06105 | 0.0843 | 0.0659 |
| | 1/10× | 0.045 | 0.048 | 0.04825 | 0.05065 | 0.0507 |
| SUZ12 | 1× | 0.06685 | 0.0686 | 0.05895 | 0.0783 | 0.09415 |
| | 1/10× | 0.0482 | 0.04985 | 0.04625 | 0.0473 | 0.05395 |
| IgG | 1/100 | 0.04665 | 0.68475 | 0.60815 | 0.79465 | 0.8879 |
| | 1/1000 | 0.0457 | 0.1914 | 0.17775 | 0.4512 | 0.52185 |
| [IgG] | | | 1 | 1 | 7 | 8 |
| μg/ml | | | | | | |

TABLE 2
MRPL11 antibody

| | sup | vector | H2AL | H2AL | L2AH | L2AH | |
|---|---|---|---|---|---|---|---|
| Antigen | dilution | only | #1 | #2 | #4 | #5 | H + L + ve |
| Erk2p | 1× | 0.05035 | 0.0534 | 0.05235 | 0.0663 | 0.0671 | 0.05755 |
| | 1/10 | 0.05075 | 0.04785 | 0.0482 | 0.06205 | 0.0483 | 0.0629 |
| MRPL11 | 1× | 0.04585 | 1.0101 | 1.057 | 0.97275 | 0.99825 | 0.9711 |
| | 1/10× | 0.045 | 0.97165 | 1.03965 | 1.14145 | 0.98965 | 1.0146 |
| SUZ12 | 1× | 0.06685 | 0.0487 | 0.0489 | 0.0542 | 0.0585 | 0.04745 |
| | 1/10× | 0.0482 | 0.0461 | 0.04455 | 0.04855 | 0.0529 | 0.04845 |
| IgG | 1/100 | 0.04665 | 0.6316 | 0.63275 | 0.8523 | 0.7972 | 0.8215 |
| | 1/1000 | 0.0457 | 0.243 | 0.20685 | 0.51115 | 0.45605 | 0.5336 |
| [IgG] | | | 2 | 2 | 9 | 8 | 10 |
| μg/ml | | | | | | | |

TABLE 3
SUZ12 antibody

| | sup | vector | H2AL | H2AL | L2AH | L2AH | |
|---|---|---|---|---|---|---|---|
| Antigen | dilution | only | #1 | #2 | #1 | #2 | H + L + ve |
| Erk2P | 1× | 0.05035 | 0.16415 | 0.1195 | 0.1093 | 0.1006 | 0.0954 |
| | 1/10× | 0.05075 | 0.0531 | 0.0662 | 0.0512 | 0.051 | 0.05475 |
| MRPL11 | 1× | 0.04585 | 0.08885 | 0.09725 | 0.08325 | 0.08235 | 0.09415 |
| | 1/10× | 0.045 | 0.05645 | 0.05205 | 0.0563 | 0.0485 | 0.0664 |
| SUZ12 | 1× | 0.06685 | 1.00675 | 1.01385 | 1.00525 | 1.0495 | 1.0243 |
| | 1/10× | 0.0482 | 1.09475 | 1.0208 | 1.1481 | 1.09315 | 0.7872 |
| IgG | 1/100 | 0.04665 | 0.83135 | 0.8156 | 0.84795 | 0.8592 | 0.9566 |
| | 1/1000 | 0.0457 | 0.29055 | 0.2636 | 0.5992 | 0.53825 | 0.59975 |
| [IgG] | | | 4 | 3 | 10 | 9 | 9 |
| μg/ml | | | | | | | |

TABLE 4
Her2 Antibody

| | sup | Empty | H2AL | H2AL | H2AL | H2AL | L2AH | L2AH | L2AH | L2AH | H + |
|---|---|---|---|---|---|---|---|---|---|---|---|
| Antigen | dilution | vector | pTT-1 | pTT-5 | pCDNA-3 | pCDNA-4 | pTT-1 | pTT-2 | pCDNA-1 | pCDNA-2 | L + ve |
| Her2 | 1× | 0.0511 | 1.1243 | 1.23975 | 1.0515 | 0.79735 | 1.2133 | 1.2075 | 0.5364 | 0.49905 | 1.27035 |
| | 1/10 | 0.05305 | 1.16255 | 1.1588 | 0.49935 | 0.32315 | 1.19295 | 1.12855 | 0.1378 | 0.13235 | 1.23275 |
| MRPL11 | 1× | 0.05345 | 0.05145 | 0.0513 | 0.04745 | 0.04795 | 0.06 | 0.0547 | 0.04585 | 0.0463 | 0.0475 |
| | 1/10 | 0.04885 | 0.04565 | 0.04685 | 0.0456 | 0.04585 | 0.0488 | 0.05085 | 0.04595 | 0.04595 | 0.045 |
| IgG | 1/100 | 0.055 | 0.8933 | 0.90005 | 0.16485 | 0.12655 | 1.20015 | 1.30985 | 0.07335 | 0.07155 | 1.27445 |
| | 1/1000 | 0.0541 | 0.2653 | 0.26915 | 0.0627 | 0.0549 | 0.42565 | 0.4553 | 0.0537 | 0.05425 | 0.41895 |
| [IgG] | | | 0.8 | 1.4 | — | — | 3.4 | 3.1 | — | — | 3.2 |
| pg/ml | | | | | | | | | | | |

For each antibody, as shown in Tables 1-4, the secreted IgG displayed specific binding to its cognate target antigen without any non-specific binding to any other antigen tested, regardless of the order of the light and heavy chains flanking the 2A peptide. The antigen-specific signal levels for the undiluted and 10-fold supernatant dilution were saturated, and thus a ten-fold dilution did not reduce the obtained value. The total IgG ELISA, which tested the supernatants at 100-fold and 1000-fold dilution due to its high sensitivity, shows a qualitative difference between the H-2A-L and L-2A-H configurations. In all cases, L-2A-H configuration expressed as well as the positive control (i.e., transfection with two plasmids—one encoding the L chain and the other encoding the H chain), and the levels of secretion (into supernatant) of antibody encoded by the L-2A-H constructs were 3 to 5-fold higher than those encoded by the H-2A-L constructs. This indicates that translation of each chain from the L-2A-H expression cassette message is as efficient as each chain being expressed as two independent transcripts from two separate vectors (and thus each H and L chain driven by its own promoter).

### Example 6

### Detection of IgG in Supernatant and Cell Lysate by Western Blotting

IgG in total cell lysate and supernatant of transfected HEK293 cells was detected by Western blotting. 10% volume of total cell lysate and 0.5% volume of total supernatant were resuspended in 1×Laemmli buffer with 42 mM DTT and boiled for 5 minutes. The chromosomal DNA was shredded in the lysate using Qiashredder (Qiagen) prior to boiling. The samples were loaded and run on a 4-20% gradient Tris-glycine polyacrylamide gel (Invitrogen), then transferred onto a nitrocellulose membrane (Whatman). The nitrocellulose membrane was blocked with 5% milk in phosphate-buffered saline (PBS) for 30 minutes, then incubated with either HRP-conjugated anti-rabbit IgG antibody (Cell Signaling Technology #7074) diluted 1000-fold in 5% milk in TBS for 1 hour at room temperature, or with anti-rabbit kappa chain antibody (Cell Signaling Technology, Inc., product #3677) diluted 1000-fold in 5% BSA in TBS overnight at 4° C. followed by an incubation with HRP-conjugated anti-mouse IgG antibody (Cell Signaling Technology, Inc., product #7076) diluted 1000-fold in 5% milk in TBS for 1 hour at room temperature. The blots were washed 4 times in TBS-T, then immersed in chemiluminescence peroxidase substrate (Cell Signaling Technology, Inc.) and exposed to film (Kodak) for detection of signal.

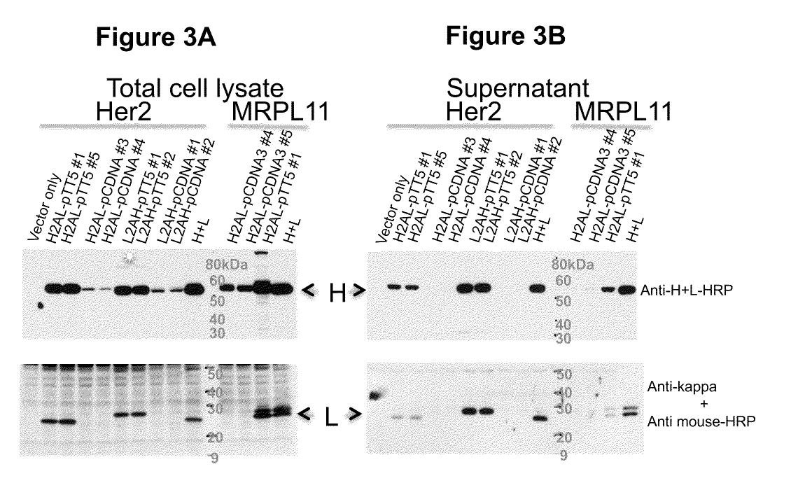

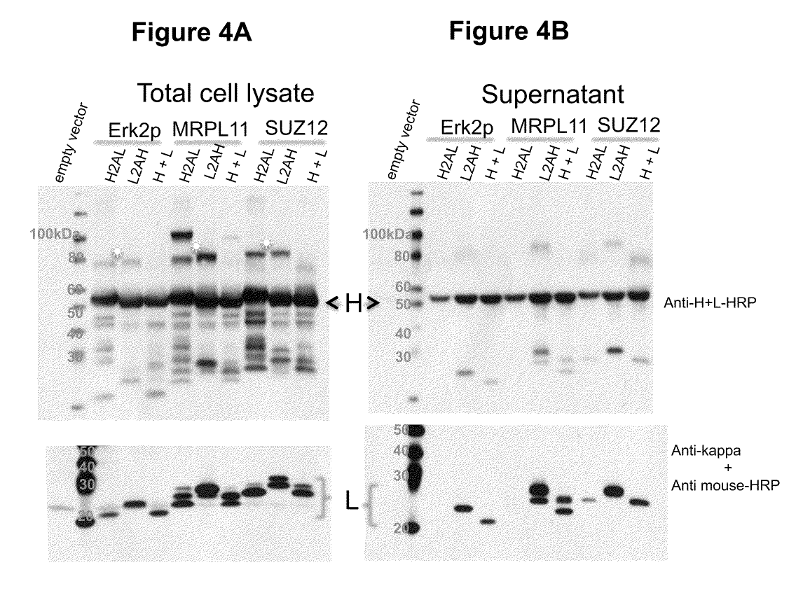

Similar levels of heavy and light chains were detected intracellularly for both H-2A-L and L-2A-H configurations (see FIGS. 3A and 4A), even though the levels of secreted total IgG was much greater for the latter (see FIGS. 3B and 4B), thus indicating that IgG expressed from the L-2A-H configuration is secreted more efficiently than from the H-2A-L configuration. The light chains of antibodies in the L-2A-H configuration, both intracellular and secreted, display a slower mobility due to the higher molecular weight from the addition of 17 amino acids on the C-termini. Full-length H-2A-L or L-2A-H translation product with a mobility of approximately 80 kDa was detected (indicated by a star on FIGS. 3A and 4A). This fusion protein is only expressed in the H-2A-L or L-2A-H constructs but not in the H+L (when each chain is expressed from its own promoter) and is mostly retained in the cells. This indicates that translation does not always pause and terminate after the T2A peptide and instead can continue to synthesize the full-length fusion protein. Samples shown in FIGS. 3A, 3B, 4A, and 4B are from the same experiment as those in Tables 1, 2, 3, and 4.

### Example 7

### Light Chain-Protease Recognition Site-2A-Heavy Chain Nucleic Acid Cassette

In this example, a nucleic acid encoding proteolytic cleavage site is introduced into the nucleic acid cassette of the invention. In this example, a site recognized by the furin protease is inserted into the nucleic acid cassette of the invention.

Furin is a ubiquitous subtilisin-related serine protease that is expressed in almost all cell types and which cleaves after the last amino acid in the following sequence: Arg-X-X-Arg (SEQ ID NO: 7; where X can be any amino acid), such as Arg-X-Arg-Arg (SEQ ID NO: 9) or Arg-X-Lys-Arg (SEQ ID NO: 8).

Using standard molecular biology methods (see, e.g., Ausubel et al., supra), the MRPL11 IgG-encoding nucleic acid cassette described in Example 1 is modified to encode a furin recognition site after the constant region of the L chain and before the 2A sequence. The resulting amino acid sequence encoded by the nucleic acid sequence of this example is set forth below:

MRPL11 IgG L-furin recognition site-2A-H cassette amino acid sequence
(SEQ ID NO: 27)
M D M R A P T Q L L G L L L L W L P G A T F A\hat{ }Q V L T Q T P S P V S A A V G N
T V T I N C Q A S Q S V R D N N Y L S W Y Q Q K P G Q P P K L L I Y R A S T L E
S G V P S R F K G N G S G T Q F T L T I S D L E C D D A A T Y Y C Q G G Y G G
N F F P F G G G T E V V V K(G D P V A P T V L I F P P A A D Q V A T G T V T I V
C V A N K Y F P D V T V T W E V D G T T Q T T G I E N S K T P Q N S A D C T Y
N L S S T L T L T S T Q Y N S H K E Y T C K V T Q G T T S V V Q S F N R G D C)
R X R R E G R G S L L T C G D V E E N P G_P M E T G L R W L L L V A V L K G
V Q C\hat{ }Q S V E E S G G R L V K P D E T L T I T C T V S G I D L N N N A M G W V
R Q A P G E G L E Y I G F I G G S G A T Y Y S T W A K G R F T I S K S S T T V D
L M I T S P T T E D T A T Y F C A R Y A G S G S F D F S G P G T L V T V S L(G Q
P K A P S V F P L A P C C G D T P S S T V T L G C L V K G Y L P E P V T V T W N
S G T L T N G V R T F P S V R Q S S G L Y S L S S V V S V T S S S Q P V T C N V
A H P A T N T K V D K T V A P S T C S K P T C P P P E L L G G P S V F I F P P K P
K D T L M I S R T P E V T C V V V D V S Q D D P E V Q F T W Y I N N E Q V R T
A R P P L R E Q Q F N S T I R V V S T L P I A H Q D W L R G K E F K C K V H N K
A L P A P I E K T I S K A R G Q P L E P K V Y T M G P P R E E L S S R S V S L T C
M I N G F Y P S D I S V E W E K N G K A E D N Y K T T P A V L D S D G S Y F L
Y S K L S V P T S E W Q R G D V F T C S V M H E A L H N H Y T Q K S I S R S P
G K)

Note that in the above amino acid sequence, the predicted leader cleavage sites are indicated with a “̂” symbol, the CDRs are all underlined, the constant region is placed in parentheses, the T2A sequence is bolded (where the “_” symbol indicates the translational skip within the T2A sequence), and the furin protease recognition site is underlined and bolded, where X is any amino acid.

The nucleic acid cassette encoding the above MRPL11 IgG L-furin recognition site-2A-H cassette amino acid sequence is inserted into an expression cloning vector (e.g., pcDNA3) which used to transfect HEK293T cells according to the methods described in the above examples. Since furin is expressed in HEK293T cells, the 2A peptide sequences are cleaved off the C terminus of the light chain portion of the encoded antibody.

### Example 8

### Assembly of a Light Chain-2A-Heavy Chain Nucleic Acid Cassette within a Vector

In this example, the components of the nucleic acid cassette are designed in a working vector prior to transferring the finished L2AH cassette, in its entirety, from the working vector to an expression vector.

The working vector is the puc9 vector (commercially available from the American Type Culture Collection, Manassas, Va.). The cloning sites on the puc9 vector used to insert components of the cassette are HindIII and BamHI.

In this example, the components of the cassette are A′-A-B-C′-C, where A′ encodes the light chain leader peptide sequence, A encodes the light chain, B encodes a 2A peptide, C′ encodes the heavy chain leader peptide sequence; and C encodes the heavy chain. The antibody described in this example is encoded by a hybridoma cell line. This example describes the process by which the nucleotide sequences encoding the chains of the antibody secreted by the hybridoma cell are isolated and used to make a nucleic acid cassette of the invention.

The light chain leader peptide and the expressed light chain (i.e., components A′-A) in this example has the following nucleotide sequence:

(SEQ ID NO: 28)

ATGGACATGAGGGCCCCCACTCAGCTGCTGGGGCTCCTGCTGCTCTGGC
TCCCAGGTGCCACATTTGCCCAAGTGCTGACCCAGACTCCATCCCCCG
TGTCTGCAGCTGTGGGAAACACAGTCACCATCAATTGCCAGGCCAGTCA
GAGTGTTCGTGATAATAACTACTTATCCTGGTATCAGCAGAAACCAGGTC
AGCCTCCCAAGCTCCTGATCTACAGGGCATCCACTCTGGAATCTGGGGT
CCCATCGCGGTTTAAAGGCAATGGATCTGGGACACAATTCACTCTCACC
ATCAGCGACCTGGAGTGTGACGATGCTGCCACTTACTATTGTCAAGGCG
GTTATGGTGGGAATTTTTTTCCTTTCGGCGGAGGGACCGAGGTGGTGGTC
AAAGGTGATCCAGTTGCACCTACTGTCCTCATCTTCCCACCAGCTGCTG
ATCAGGTGGCAACTGGAACAGTCACCATCGTGTGTGTGGCGAATAAAT
ACTTTCCCGATGTCACCGTCACCTGGGAGGTGGATGGCACCACCCAA
ACAACTGGCATCGAGAACAGTAAAACACCGCAGAATTCTGCAGATTGT
ACCTACAACCTCAGCAGCACTCTGACACTGACCAGCACACAGTACAA
CAGCCACAAAGAGTACACCTGCAAGGTGACCCAGGGCACGACCTCA
GTCGTCCAGAGCTTCAATAGGGGTGACTGTTAG

The heavy chain leader peptide and the expressed heavy chain (i.e., components C′-C in the nucleic acid cassette) in this example has the following nucleotide sequence:

(SEQ ID NO: 29)
ATGGAGACTGGGCTGCGCTGGCTTCTCCTGGTCGCTGTGCTCAAAGGTGTCCAGTGTCA
GTCGGTGGAGGAGTCCGGGGGTCGCCTGGTCAAGCCTGACGAAACCCTGACAATCACCT
GCACAGTCTCTGGAATCGACCTCAATAACAATGCAATGGGCTGGGTCCGCCAGGCTCCA
GGGGAGGGGCTGGAATACATCGGATTCATTGGTGGGAGTGGTGCCACATACTACTCGAC
CTGGGCGAAAGGCCGGTTCACCATCTCCAAGTCCTCGACCACGGTGGATCTGATGATCA
CCAGTCCGACAACCGAGGACACGGCCACCTATTTCTGTGCCAGATATGCTGGTAGTGGT
TCTTTTGACTTCTCGGGCCCAGGCACCCTGGTCACCGTCTCCTTGGGGCAACCTAAGGC
TCCATCAGTCTTCCCACTGGCCCCCTGCTGCGGGGACACACCCAGCTCCACGGTGACCC
TGGGCTGCCTGGTCAAAGGCTACCTCCCGGAGCCAGTGACCGTGACCTGGAACTCGGGC
ACCCTCACCAATGGGGTACGCACCTTCCCGTCCGTCCGGCAGTCCTCAGGCCTCTACTC
GCTGAGCAGCGTGGTGAGCGTGACCTCAAGCAGCCAGCCCGTCACCTGCAACGTGGCCC
ACCCAGCCACCAACACCAAAGTGGACAAGACCGTTGCGCCCTCGACATGCAGCAAGCCC
ACGTGCCCACCCCCTGAACTCCTGGGGGGACCGTCTGTCTTCATTTTCCCCCCAAAACC
CAAGGACACCCTCATGATCTCACGCACCCCCGAGGTCACATGCGTGGTGGTGGACGTGA
GCCAGGATGACCCCGAGGTGCAGTTCACATGGTACATAAACAACGAGCAGGTGCGCACC
GCCCGGCCGCCGCTACGGGAGCAGCAGTTCAACAGCACGATCCGCGTGGTCAGCACCCT
CCCCATCGCGCACCAGGACTGGCTGAGGGGCAAGGAGTTCAAGTGCAAAGTCCACAACA
AGGCACTCCCGGCCCCCATCGAGAAAACCATCTCCAAAGCCAGAGGGCAGCCCCTGGAG
CCGAAGGTCTACACCATGGGCCCTCCCCGGGAGGAGCTGAGCAGCAGGTCGGTCAGCCT
GACCTGCATGATCAACGGCTTCTACCCTTCCGACATCTCGGTGGAGTGGGAGAAGAACG
GGAAGGCAGAGGACAACTACAAGACCACGCCGGCCGTGCTGGACAGCGACGGCTCCTAC
TTCCTCTACAGCAAGCTCTCAGTGCCCACGAGTGAGTGGCAGCGGGGCGACGTCTTCAC
CTGCTCCGTGATGCACGAGGCCTTGCACAACCACTACACGCAGAAGTCCATCTCCCGCT
CTCCGGGTAAATGA

The 2A peptide (i.e., component B) in this example will have the following nucleotide sequence: gacgtggaggagaatcccggccct (SEQ ID NO: 30).

To generate the cassette, the B′-B-C components are generated first and are inserted into the puc9 vector.

To do this, the following PCR primers are generated. 5′ agtggatccgacgtggaggagaatcccggccctATGGAGACTGG3′ (SEQ ID NO: 31; where the BamHI recognition site is underlined and the nucleotide sequence encoding the 2A peptide is italicized and bolded, and the nucleotide sequence encoding the heavy chain is capitalized).

5′ taggctgagTCATTTACCCGGAGA3′ (SEQ ID NO: 32; where the PstI recognition site is underlined and the antisense of the nucleotide sequence encoding the heavy chain is capitalized)

mRNA is isolated from the hybridoma cell line, and subjected to PCR using the above two PCR primers. The resulting PCR product is electrophoretically resolved on a low-melting point agarose gel, purified from the gel, and incubated at 37° C. with PstI and BamHI restriction endonucleases in buffer supplied by the manufacturer (New England Biolabs, Ipswich, Mass.).

A puc9 vector is digested with PstI and BamHI. The digested DNAs are electrophoretically resolved in low melting point agarose, and the vector fragment (i.e., the larger fragment) from the digested puc9 is ligated to the digested PCR product. The ligation is used to transform competent E. coli, and the resulting cells are plated onto LB agar plates containing ampicillin. Positive clones are picked and expanded, minipreps are performed, and ligated vector is isolated. The vector's insert is then sequenced (e.g., using a sequencing primer that hybridizes upstream of the BamHI site in puc9).

The A′-A components of the cassette is next constructed. A forward PCR primer is generated that adds a HindIII recognition site at the 5′ end of the above mentioned A′ sequence. For example, the PCR primer may have the following sequence: 5′ gggaagcttATGGACATGAGGG 3′ (SEQ ID NO: 33; where the HindIII recognition site is underlined and the nucleotide sequence encoding the light chain is capitalized.)

The reverse primer is constructed to add a PstI recognition site at the 3′ end of the A sequence, but removing the stop codon (in this case TGA) from the sequence. As an example, the reverse primer will have the sequence: 5′ ggctgcagACAGTCACCCCTAT(SEQ ID NO: 34; where the PstI recognition site is underlined and the nucleotide sequence encoding the 2A peptide is italicized and bolded, and the nucleotide sequence encoding the heavy chain is capitalized).

The heavy chain is PCR amplified from mRNA from the hybridoma, and digested with HindIII and PstI. The B-C containing puc9 vector (i.e., the vector containing the nucleotide sequence encoding the 2A peptide and the light chain) is similarly digested with HindIII and PstI, and the digested vector fragment is ligated with the digested PCR fragment. The ligation mixture is used to transform E. coli, and positive clones (i.e., ampicillin resistant) are screened by digesting miniprep DNA with restriction endonucleases chosen to identify those vectors having the heavy chain inserted in the correct orientation.

After a positive clone is identified and sequenced, the entire cassette (which now runs from HindIII at the 5′ end to BamHI at the 3′ end) is excised by digesting the puc9 vector containing the cassette with HindIII and BamHI and isolating the insert fragment from the vector fragment.

The pcDNA3.1 vector is purchased from Invitrogen (Carlsbad, Calif.) and is digested with HindIII and BamHI. The vector fragment is ligated to the insert fragment (containing the nucleic acid cassette), and at least one positive clone is isolated, expanded, and the supercoiled plasmid DNA is purified.

COS cells are transfected using DEAE-dextran with the cassette/pcDNA3 vector linearized by digesting with a restriction endonuclease that does not recognize a site either in the cassette, in the neomycin resistance gene, or in either the promoter for the cassette or the promoter for the neo gene. (For example, PvuI is used to linearize the cassette/pcDNA3.1 construct. Transfected cells are selected in G418-containing media and the cells are cloned by limiting dilution. Expanded clones are then screened for high secretion rates of the antibody, and the ability of the secreted antibody to bind to its target molecule (using, for example, an ELISA assay as described in Example 3 above).

### Example 9

### A Stable Cell Line Expressing the IgG Encoded by the L-2A-H Nucleic Acid Cassette

In this example, an episomally replicable expression vector containing components of the nucleic acid cassette of the invention is provided.

The expression plasmid pCEP4 is purchased from Invitrogen (Carlsbad, Calif.) and is digested with HindIII and BamHI. The digested plasmid is electrophoretically resolved and the vector band purified. The HindIII to BamHI nucleic acid cassette described in Example 8 is excised from puc9 and ligated into the digested pCEP4 vector. Transformed E. coli are selected on ampicillin-containing agar plates and positive clones sequenced. Plasmid DNA is purified from the selected positive clones and used to transfect Jurkat T cells. Since pCEP4 contains both EBNA1 and oriP (the origin of replication for EBV), it is capable of episomally replicating in the cell, and so does not require stable integration. Jurkat T cells are transfected using electroporation.

To generate stable cells, the transfected cells are selected in hygromycin-containing media. The hygromycin resistant cells are cloned by limiting dilution.

Note that if cells transiently expressing the antibody encoded by the nucleic acid cassette are desired, the cells are not selected or cloned.

Conditioned media from the cells is collected and antibody in the media is enriched using, for example, the ability of the Fc portion of antibody to bind protein A sepharose.

### Example 10

### A Vector Containing Nucleic Acid Encoding the 2A Peptide

In this example, a cloning vector is generated containing the nucleic acid encoding the 2A peptide component of the cassette within the polylinker of the plasmid. This vector, together with instructions for inserting nucleic acid sequences encoding the heavy chain and the light chain of an antibody of interest and, optionally, with PCR primers for facilitating cloning in the H and L chain sequences, may be sold as a kit.

For example, the puc9 plasmid is used as the cloning vector. The 2A peptide in this example has the amino acid sequence DVEENPGP (SEQ ID NO: 35). Given the degeneracy of the genetic code, numerous different nucleotide sequences encode this 2A peptide sequence. For example, the following nucleotide sequence 5′Gacgtcgaagagaacccagggccc3′ (SEQ ID NO: 36) is used. The 5′ end of this sequence is an AatII recognition site, while the 3′ end of this sequence is an ApaI site.

Thus, the cloning sites of the cassette within the polylinker of the puc9 plasmid may be: (puc9 backbone sequences)-AAGCTT (HindIII site)-(random sequences)-Gacgtc(AatII site)-gaagagaaccca (SEQ ID NO: 37)-gggccc(ApaI site)-(random sequences)-GGATCC (BamHI site)-(puc9 sequences).

A map of this cloning vector will be provided with the kit (full length sequence and map of puc9 is available from the American Type Culture Collection), and instructions such that the practitioner will be able to design PCR primers which add the appropriate restriction endonuclease recognition site to the amplified sequence to facilitate insertion of nucleic acids encoding the H chain and L chain of the antibody into the vector. In the above-example, the H chain-encoding nucleic acid sequence will be inserted into the ApaI to BamHI insertion site, and the L chain-encoding nucleic acid sequence will be inserted into the HindIII to AatII site. Care is taken to ensure that the inserted sequences are in frame with the 2A peptide, such that entire nucleic acid sequence contained within the HindIII and the BamHI sites are in a single open reading frame and, but for the translation “stop” signal by the 2A peptide, would be translated as a single polypeptide. Note that the puc9 vector backbone can also be substituted with an expression cloning plasmid (e.g., the pcDNA3.1 vector described above).

Where the practitioner desires to obtain a cassette encoding a secreted antibody, the cloning vector can also include nucleic acid sequences encoding a leader peptide upstream of the HindIII site for the first chain and immediately after the ApaI site for the second chain (a new restriction endonuclease site may be engineered at the 3′ end of the leader peptide to facilitate insertion of the second chain).

The kit may further include PCR primers designed to facilitate insertion of the nucleotide sequences encoding the antibody of interest into the cloning vector. In some embodiments, because of the huge diversity in the antigen binding domain portion of an antibody chain among different species (e.g., difference between mice and humans), however, provision of such primers with the kit may limit the number of species from which antibodies of interest are derived that the practitioner is able to insert into the cloning vector.

### Example 11

### Construction of a Mouse Light Chain-2A-Heavy Chain Nucleic Acid Cassette

The light chain-2A-heavy chain (L-2A-H) cassette encoding a mouse antibody will be assembled as a single molecule of DNA using two steps of polymerase chain reaction (PCR).

Briefly, the first step will consist of two independent reactions, one that will amplify the light chain (or 5′ piece of the cassette) with Primer A and Primer B from a template encoding the full length light chain sequence including the light chain leader sequence, and the other that will amplify the heavy chain (or 3′ end of the cassette) with Primer C and Primer D from a template encoding the full length heavy chain sequence including the heavy chain leader sequence. The sequences of Primers A-D will be as follows (where the sequences that will be derived from murine sequences are underlined):

(SEQ ID NO: 38)

Primer A: 5′ GTCGTCAAGCTTATGAGGGCCCCTGCTCAGATT 3′

(SEQ ID NO: 39)

Primer B: 5′
CTCCACGTCACCGCATGTTAGAAGACTTCCTCTGCCCTCACACTCATTC
CTGTTGAAGCT 3′

(SEQ ID NO: 40)

Primer C: 5′
TCTTCTAACATGCGGTGACGTGGAGGAGAATCCCGGCCCTATGGCTTG
GGTGTGGACCTTG 3′

(SEQ ID NO: 41)

Primer D: 5′ ATAAGAATGCGGCCGCTATCATTTACCAGGAGAG
TGGGA 3′

The template DNA to be used to construct the nucleic acid cassette will be derived from the following sources: heavy chain variable region from GenBank AB016619.1, IgG1 isotype heavy chain constant region from GenBank AK144480.1, light chain variable region from GenBank AB016620.1 and kappa chain isotype constant region from GenBank BC091750.1. The variable region of the expressed recombinant antibody is derived from FU-MK1 (see Arakawa F et al., “cDNA sequence analysis of monoclonal antibody FU-MK-1 specific for a transmembrane carcinoma-associated antigen, and construction of a mouse/human chimeric antibody”. Hybridoma. 18(2):131-138 (April 1999)), and is specific to a human gastric adenocarcinoma transmembrane antigen, GA733-2*. The methodology for construction of the mouse L-2A-H IgG cassette will be conducted as described for the rabbit L-2A-H IgG cassette described in Example 1.

32

The nucleotide sequence of the resulting mouse light chain 2A heavy chain cassette will be:

(SEQ ID NO: 42)
ATGAGGGCCCCTGCTCAGATTCTTGGCTTCTTGTTGCTCTGGTTTCCAGGTATTAGATGT
GACATCAAGATGACCCAGTCGCCATCCTCCTTATCTGCCTCTCTGGGAGAAAG
AGTCAGTCTCACTTGTCGGGCAAGTCAGGAAATTAGTGGTTACTTAAGCTG
GCTTCAGCAAAAACCAGATGGAACTGTTAAACGCCTGATCTACGCCG
CATCCACTTTACATTCTGGTGTCCCAAAAAGGTTCAGTGGCAGTAGGTCTGGGTCAGA
CTATTCTCTCACCATCAGCAGCCTTGAGTCTGACGATTTTGCAGACTATTACTGT
CTACAGTATGCTAGTGATCCGTGGACGTTCGGTGGAGGCACCAAGCTGGA
AATCAAACGGGCTGATGCTGCACCAACTGTATCCATCTTCCCACCATCCAGTGAGCAGT
TAACATCTGGAGGTGCCTCAGTCGTGTGCTTCTTGAACAACTTCTACCCCA
AAGACATCAATGTCAAGTGGAAGATTGATGGCAGTGAACGACAAAATGG
CGTCCTGAACAGTTGGACTGATCAGGACAGCAAAGACAGCACCTAC
AGCATGAGCAGCACCCTCACGTTGACCAAGGACGAGTATGAACGACATAAC
AGCTATACCTGTGAGGCCACTCACAAGACATCAACTTCACCCATCGTCAAGA
GCTTCAACAGGAATGAGTGTGAGGGCAGAGGAAGTCTTCTAACATGCGGTGA
CGTGGAGGAGAATCCCGGCCCTATGGCTTGGGTGTGGACCTTGCTATTCCTGA
TGGCAGCTGCCCAAAGTATCCAAGCACAGATCCAGTTGGTGCAGTCTGGACC
TGAGCTGAAGAAGCCTGGAGAGACAGTCAAGATCTCCTGCAAGACTTCTGGT
TATACCTTCACAGACTATTCAATGCACTGGGTGAAGCAGGCTCCAGGAAAGG
GTTTAAAGTGGATGGGCTGGATAAACACTGAGACTGGTGGGCCAACATATGC
AGATGACTTCAAGGGACGGTTTGCCTTCTCTTTGGAAACCTCTGCCAGCACTG
CCTATTTGCAGATCAACAACCTCAAAAATGAGGACACGGCTACATATTTCTGT
GCTAGAACTTCCGTCTACTGGGGCCAAGGCACCACTCTCACAGTCTCCTCAGC
CAAAACGACACCCCCATCTGTCTATCCACTGGCCCCTGGATCTGCTGCCCAAA
CTAACTCCATGGTGACCCTGGGATGCCTGGTCAAGGGCTATTTCCCTGAGCCA
GTGACAGTGACCTGGAACTCTGGATCCCTGTCCAGCGGTGTGCACACCTTCCC
AGCTGTCCTGCAGTCTGACCTCTACACTCTGAGCAGCTCAGTGACTGTCCCCT
CCAGCACCTGGCCCAGCCAGACCGTCACCTGCAACGTTGCCCACCCGGCCAG
CAGCACCAAGGTGGACAAGAAAATTGTGCCCAGGGATTGTGGTTGTAAGCCT
TGCATATGTACAGTCCCAGAAGTATCATCTGTCTTCATCTTCCCCCCAAAGCC
CAAGGATGTGCTCACCATTACTCTGACTCCTAAGGTCACGTGTGTTGTGGTAG
ACATCAGCAAGGATGATCCCGAGGTCCAGTTCAGCTGGTTTGTAGATGATGT
GGAGGTGCACACAGCTCAGACGAAACCCCGGGAGGAGCAGATCAACAGCAC
TTTCCGTTCAGTCAGTGAACTTCCCATCATGCACCAGGACTGGCTCAATGGCA
AGGAGTTCAAATGCAGGGTCAACAGTGCAGCTTTCCCTGCCCCCATCGAGAA
AACCATCTCCAAAACCAAAGGCAGACCGAAGGCTCCACAGGTGTACACCATT
CCACCTCCCAAGGAGCAGATGGCCAAGGATAAAGTCAGTCTGACCTGCATGA
TAACAAACTTCTTCCCTGAAGACATTACTGTGGAGTGGCAGTGGAATGGGCA
GCCAGCGGAGAACTACAAGAACACTCAGCCCATCATGGACACAGATGGCTCT
TACTTCGTCTACAGCAAGCTCAATGTGCAGAAGAGCAACTGGGAGGCAGGAA
ATACTTTCACCTGCTCTGTGTTACATGAGGGCCTGCACAACCACCATACTGAG
AAGAGCCTCTCCCACTCTCCTGGTAAATGA

The amino acid sequence of the resulting mouse light chain 2A heavy chain cassette will be:

(SEQ ID NO: 43)
MRAPAQILGFLLLWFPGIRC\hat{ }DIKMTQSPSSLSASLGERVSLTCRASQEISGYLSW
LQQKPDGTVKRLIYAASTLHSGVPKRFSGSRSGSDYSLTISSLESDDFADYYCL
QYASDPWTFGGGTKLEIK(RADAAPTVSIFPPSSEQLTSGGASVVCFLNNFYPKDINVK
WKIDGSERQNGVLNSWTDQDSKDSTYSMSSTLTLTKDEYERHNSYTCEATH
KTSTSPIVKSFNRNEC)EGRGSLLTCGDVEENPGPMAWVWTLLFLMAAAQSIQ
A\hat{ }QIQLVQSGPELKKPGETVKISCKTSGYTFTDYSMHWVKQAPGKGLKWMGWI
NTETGGPTYADDFKGRFAFSLETSASTAYLQINNLKNEDTATYFCARTSVYWGQ
GTTLTVSS(AKTTPPSVYPLAPGSAAQTNSMVTLGCLVKGYFPEPVTVTWNSGSL
SSGVHTFPAVLQSDLYTLSSSVTVPSSTWPSQTVTCNVAHPASSTKVDKKIVPRD
CGCKPCICTVPEVSSVFIFPPKPKDVLTITLTPKVTCVVVDISKDDPEVQFSWFVD
DVEVHTAQTKPREEQINSTFRSVSELPIMHQDWLNGKEFKCRVNSAAFPAPIEKTI
SKTKGRPKAPQVYTIPPPKEQMAKDKVSLTCMITNFFPEDITVEWQWNGQPAEN
YKNTQPIMDTDGSYFVYSKLNVQKSNWEAGNTFTCSVLHEGLHNHHTEKSLSHSPGK)

Note that in the above amino acid sequence, the predicted leader cleavage sites are indicated with a “̂” symbol, the CDRs are all underlined, the constant region is placed in parentheses, and the T2A sequence is bolded (where the “_” symbol indicates the translational skip within the T2A sequence).

### Example 12

### Construction of a Human Light Chain-2A-Human Heavy Chain Nucleic Acid Cassette

The light chain-2A-heavy chain (L-2A-H) cassette encoding a human antibody will be assembled as a single molecule of DNA using two steps of polymerase chain reaction (PCR).

Briefly, the first step will consist of two independent reactions, one that will amplify the light chain (or 5′ piece of the cassette) with Primer A and Primer B from a template encoding the full length light chain sequence including the light chain leader sequence, and the other that will amplify the heavy chain (or 3′ end of the cassette) with Primer C and Primer D from a template encoding the full length heavy chain sequence including the heavy chain leader sequence. The sequences of Primers A-D will be as follows (where the sequences that will be derived from human sequences are underlined):

(SEQ ID NO: 44)

Primer A: 5′ GTCGTCAAGCTTATGGAAACCCCAGCGCCAGT 3′

(SEQ ID NO: 45)

Primer B: 5′
CTCCACGTCACCGCATGTTAGAAGACTTCCTCTGCCCTCGCACTCTCC
CCTGTTGCTCTT 3′
(SEQ ID NO: 46)
Primer C: 5′
TCTTCTAACATGCGGTGACGTGGAGGAGAATCCCGGCCCTATGGACT
GCACCTGGAGGAT 3′
(SEQ ID NO: 47)
Primer D: 5′ ATAAGAATGCGGCCG CTACTATTTACCCGGAGACA
GGGA 3′

The template DNA to be used to construct the nucleic acid cassette is cDNA generated from RNA isolated from an Epstein Barr Virus-immortalized human B-cell culture that secretes IgG with polyreactivity. The methodology for construction of the human L-2A-H IgG cassette will be conducted as described for the rabbit L-2A-H IgG cassette described in Example 1.

The nucleotide sequence of the resulting human light chain 2A heavy chain cassette will be:

(SEQ ID NO: 48)
ATGGAAACCCCAGCGCCAGTTCTCTTCCTCCTGCTACTCTGGCTCCCAGATACCGG
AGACATCCAGATGACCCAGTCTCCATCCTCCCTGTCTGCATCTGTAGGAGACAG
AGTCACCATCACTTGCCGGGCAAGTCAGAGCATTAGCAGCTATTTAAATTGGT
ATCAGCAGAAACCAGGGAAAGCCCCTAAGCTCCTGATCTATGCTGCATCCA
GTTTGCAAAGTGGGGTCCCATCAAGGTTCAGTGGCAGTGGATCTGGGACAGA
TTTCACTCTCACCATCAGCAGTCTGCAACCTGAAGATTTTGCAACTTACTA
CTGTCAACAGAGTTACAGTACCCCCTACACTTTTGGCCAGGGGACCAAGC
TGGAGATCAAACGAACTGTGGCTGCACCATCTGTCTTCATCTTCCCGCCATCT
GATGAGCAGTTGAAATCTGGAACTGCCTCTGTTGTGTGCCTGCTGAATAACTT
CTATCCCAGAGAGGCCAAAGTACAGTGGAAGGTGGATAACGCCCTCCAATCG
GGTAACTCCCAGGAGAGTGTCACAGAGCAGGACAGCAAGGACAGCACCTAC
AGCCTCAGCAGCACCCTGACGCTGAGCAAAGCAGACTACGAGAAACACAAA
GTCTACGCCTGCGAAGTCACCCATCAGGGCCTGAGCTCGCCCGTCACAAAGA
GCTTCAACAGGGGAGAGTGCGAGGGCAGAGGAAGTCTTCTAACATGCGGTG
ACGTGGAGGAGAATCCCGGCCCTATGGACTGCACCTGGAGGATCCTCCTCTT
GGTGGCAGCAGCTACAGGCACCCACGCCGAGGTCCAGGTGCAGCTGGTGGAGTC
TGGGGGAGGCTTGGTCCAGCCTGGGGGGTCCCTGAGACTCTCCTGTGCAGCC
TCTGGATTCACCTTTAGTGATTATTGGATGAGCTGGGTCCGCCAGGCTCCAGGGAA
GGGGCTGGAGTGGGTGGCCCACATAAAGCAAGATGGAAGTGAGAAATACTAT
GTGGACTCTGTGAAGGGCCGATTCACCATCTCCCGAGACAAAGCCAAGAACTC
ACTGTATCTCCAAATGAACAGCCTGAGAGCCGAGGACACGGCCGTGTA
TTACTGTGCGAGGTGTCCGGTGCGGGAACGTGACTGGTACCGTGCGCG
TGGGGAGTACTACTACGTCTACATGGACGTCTGGGGCAAGGGGACCACGGTC
ACCGTCTCCTCAGCTTCCACCAAGGGCCCATCGGTCTTCCCCCTGGCACCCTC
CTCCAAGAGCACCTCTGGGGGCACAGCGGCCCTGGGCTGCCTGGTCAAGGAC
TACTTCCCCGAACCGGTGACGGTGTCGTGGAACTCAGGCGCCCTGACCAGCG
GCGTGCACACCTTCCCGGCTGTCCTACAGTCCTCAGGACTCTACTCCCTCAGC
AGCGTGGTGACCGTGCCCTCCAGCAGCTTGGGCACCCAGACCTACATCTGCA
ACGTGAATCACAAGCCCAGCAACACCAAGGTGGACAAGAGAGTTGAGCCCA
AATCTTGTGACAAAACTCACACATGCCCACCGTGCCCAGCACCTGAACTCCT
GGGGGGACCGTCAGTCTTCCTCTTCCCCCCAAAACCCAAGGACACCCTCATG
ATCTCCCGGACCCCTGAGGTCACATGCGTGGTGGTGGACGTGAGCCACGAAG
ACCCTGAGGTCAAGTTCAACTGGTACGTGGACGGCGTGGAGGTGCATAATGC
CAAGACAAAGCCGCGGGAGGAGCAGTACAACAGCACGTACCGTGTGGTCAG
CGTCCTCACCGTCCTGCACCAGGACTGGCTGAATGGCAAGGAGTACAAGTGC
AAGGTCTCCAACAAAGCCCTCCCAGCCCCCATCGAGAAAACCATCTCCAAAG
CCAAAGGGCAGCCCCGAGAACCACAGGTGTACACCCTGCCCCCATCCCGGGA
GGAGATGACCAAGAACCAGGTCAGCCTGACCTGCCTGGTCAAAGGCTTCTAT
CCCAGCGACATCGCCGTGGAGTGGGAGAGCAATGGGCAGCCGGAGAA
CAACTACAAGACCACGCCTCCCGTGCTGGACTCCGACGGCTCCTTCTTCCTCT
ATAGCAAGCTCACCGTGGACAAGAGCAGGTGGCAGCAGGGGAACGTCTTCTCAT
GCTCCGTGATGCATGAGGCTCTGCACAACCACTACACGCAGAAGAGCCTCTCCCTGTCTC
CGGGTAAATAG

The amino acid sequence of the resulting human light chain 2A heavy chain cassette will be:

(SEQ ID NO: 49)
METPAPVLFLLLLWLPDTG\hat{ }DIQMTQSPSSLSASVGDRVTITCRASQSISSYLNWYQQ
KPGKAPKLLIYAASSLQSGVPSRFSGSGSGTDFTLTISSLQPEDFATYYCQQSYST
PYTFGQGTKLEIK(RTVAAPSVFIFPPSDEQLKSGTASVVCLLNNFYPREAKVQ
WKVDNALQSGNSQESVTEQDSKDSTYSLSSTLTLSKADYEKHKVYACEVTHQG
LSSPVTKSFNRGEC)EGRGSLLTCGDVEENPGPMDCTWRILLLVAAATGTHA\hat{ }E
VQVQLVESGGGLVQPGGSLRLSCAASGFTFSDYWMSWVRQAPGKGLEWVAHI
KQDGSEKYYVDSVKGRFTISRDKAKNSLYLQMNSLRAEDTAVYYCARCPVRER
DWYRARGEYYYVYMDVWGKGTTVTVSS(ASTKGPSVFPLAPSSKSTSGGTAAL
GCLVKDYFPEPVTVSWNSGALTSGVHTFPAVLQSSGLYSLSSVVTVPSSSLGTQT
YICNVNHKPSNTKVDKRVEPKSCDKTHTCPPCPAPELLGGPSVFLFPPKPKDTLMI
SRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLTVDKSRWQQ
GNVFSCSVMHEALHNHYTQKSLSLSPGK)

Note that in the above amino acid sequence, the predicted leader cleavage sites are indicated with a “̂” symbol, the CDRs are all underlined, the constant region is placed in parentheses, and the T2A sequence is bolded (where the “_” symbol indicates the translational skip within the T2A sequence).

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

What is claimed is:

1. A nucleic acid cassette comprising components in the following structure in a 5′ to 3′ direction on a sense strand:
A-B-C,
wherein “A” is a nucleic acid sequence encoding at least an antigen binding domain of a light chain of a first antibody, “B” is a nucleic acid sequence encoding a 2A peptide, “C” is a nucleic acid sequence encoding at least an antigen binding domain of a heavy chain of a second antibody, and “−” is a bond selected from the group consisting of a phosphodiester bond and a phosphorothioate bond.

2. A nucleic acid cassette comprising components in the following structure in a 5′ to 3′ direction on a sense strand:
A-B-C,
wherein “A” is a nucleic acid sequence encoding a light chain of a first antibody, “B” is a nucleic acid sequence encoding a 2A peptide, “C” is a nucleic acid sequence encoding a heavy chain of a second antibody, and “−” is a bond selected from the group consisting of a phosphodiester bond and a phosphorothioate bond.

3. The nucleic acid cassette of claim 1 or 2, further comprising components in the following structure:
A!-A-B-C!-C,
wherein “A!” is a nucleic acid sequence encoding a first leader peptide, and “C!” is a nucleic acid sequence encoding a second leader peptide.

4. The nucleic acid cassette of claim 1 or 2, further comprising components in the following structure:
A-B-C-D,
wherein “D” is a nucleic acid sequence encoding a tag.

5. The nucleic acid cassette of claim 1 or 2, further comprising components in the following structure:
A-p-B-C,
wherein “p” is a nucleic acid sequence encoding a protease recognition site.

6. A nucleic acid cassette comprising components in the following structure:
A-a-B-C-c,
wherein “A” is a nucleic acid sequence encoding an antigen binding domain of a light chain of a first antibody, “a” is a nucleic acid sequence encoding a stem of a light chain of a second antibody, “B” is a nucleic acid sequence encoding a 2A peptide, “C” is a nucleic acid sequence encoding an antigen binding domain of a heavy chain of a third antibody, “c” is a nucleic acid sequence encoding a stem of a heavy chain of a fourth antibody, and “−” is a bond selected from the group consisting of a phosphodiester bond and a phosphorothioate bond.

7. The nucleic acid cassette of claim 6, further comprising components in the following structure:
A!-A-a-B-C!-C-c,
wherein “A!” is a nucleic acid sequence encoding a first leader peptide, and “C!” is a nucleic acid sequence encoding a second leader peptide.

8. The nucleic acid cassette of claim 6, further comprising components in the following structure:
A-a-B-C-c-D,
wherein “D” is a nucleic acid sequence encoding a tag.

9. The nucleic acid cassette of claim 6, further comprising components in the following structure:
A-a-p-B-C-c,
wherein “p” is a nucleic acid sequence encoding a protease recognition site.

10. The nucleic acid cassette of claim 1, 2, or 6, wherein the 2A peptide comprises an amino acid sequence selected from the group consisting of DVEXNPGP and DIEXNPGP, where X is any amino acid residue.

11. The nucleic acid cassette of claim 1, 2, or 6, wherein the 2A comprises an amino acid sequence of EGRGSLLTCGDVEENPGP.

12. The cassette of claim 1, 2, or 6, wherein the antibody is of an isotype selected from the group consisting of IgG, IgD, IgA, IgE, and IgM.

13. A vector comprising the cassette of claim 1, 2, or 6.

14. The vector of claim 13, wherein the vector is an expression vector.

15. A method for producing a recombinant antibody comprising (a) introducing the nucleic acid cassette of claim 1, 2, or 6 into a cell such that the cell expresses the nucleic acid cassette; (b) maintaining the cell of step (a) in a culture media, and isolating the antibody from the cell or the culture media of step (b).

16. A cell comprising the nucleic acid cassette of claim 1, 2, or 6.

17. The cell of claim 16, wherein the cell expresses the nucleic acid cassette.

18. A recombinant antibody produced by the cell of claim 17.

19. A kit comprising:
a first primer comprising a 5′ portion comprising a recognition site of a first restriction endonuclease and a 3′ portion that hybridizes to an antisense strand of a nucleic acid sequence encoding a leader peptide of a light chain of a first antibody;
a second primer comprising a 5′ portion comprising a nucleic acid sequence that is complementary to a first part of a 2A-peptide encoding nucleic acid sequence and a 3′ portion that hybridizes to a nucleic acid sequence encoding a constant region of a light chain of a second antibody;
a third primer comprising a 5′ portion comprising a nucleic acid sequence that encodes a second part of a 2A peptide and a 3′ portion that hybridizes to an antisense strand of a nucleic acid sequence encoding a leader peptide of a heavy chain of a third antibody;
a fourth primer comprising a 5′ portion comprising a recognition site of a second restriction endonuclease and a 3′ portion that hybridizes to a nucleic acid sequence encoding a constant region of a heavy chain of a fourth antibody; and
instructions for using the first, second, third, and fourth primers to generate a nucleic acid cassette from a sample comprising nucleic acid encoding the first antibody, the second antibody, the third antibody, and the fourth antibody.

20. The kit of claim 19, wherein the first antibody and the second antibody are the same.

21. The kit of claim 19, wherein the third antibody and the fourth antibody are the same.

22. The kit of claim 19, wherein the first antibody, second antibody, third antibody, and fourth antibody are the same.

23. The kit of claim 19, further comprising a thermostable DNA polymerase.

24. The kit of claim 19, further comprising a first restriction endonuclease and a second restriction endonuclease.

25. The kit of claim 19, wherein the first restriction endonuclease and the second restriction endonuclease are the same.

26. The kit of claim 19, further comprising a vector comprising a polylinker comprising the first restriction endonuclease recognition site and the second restriction endonuclease recognition site.

27. A method for making a nucleic acid cassette comprising
(a) amplifying a nucleic acid molecule encoding a light chain comprising a leader peptide and a constant region of a first antibody with a first primer comprising a 5′ portion comprising a recognition site of a first restriction endonuclease and a 3′ portion that hybridizes to an antisense strand of a nucleic acid sequence encoding the leader peptide of the light chain and a second primer comprising a 5′ portion comprising a nucleic acid sequence that is complementary to a first part of a 2A-peptide encoding nucleic acid sequence and a 3′ portion that hybridizes to a nucleic acid sequence encoding the constant region of the light chain;
(b) amplifying a nucleic acid molecule encoding a heavy chain comprising a leader peptide and a constant region of a second antibody with a third primer comprising a 5′ portion comprising a nucleic acid sequence that encodes a second part of a 2A peptide and a 3′ portion that hybridizes to an antisense strand of a nucleic acid sequence encoding the leader peptide of the heavy chain and a fourth primer comprising a 5′ portion comprising a recognition site of a second restriction endonuclease and a 3′ portion that hybridizes to a nucleic acid sequence encoding the constant region of the heavy chain of a third antibody;
(c) allowing the products of step (a) and step (b) to hybridize to each other; and
(d) amplifying the product of step (c) with the first primer and the fourth primer.
